(19) United States
(12) Patent Application Publication
(10) Pub. No.: US 2022/0301682 A1
(43) Pub. Date: Sep. 22, 2022

(54) Dynamically Adaptable Health Experience based on Data Triggers

(71) Applicant: Adobe Inc., San Jose, CA (US)

(72) Inventors: Kalyanaraman Balasubramaniam Krishnan, Milpitas, CA (US); Jennifer Anne Healey, San Jose, CA (US)

(73) Assignee: Adobe Inc., San Jose, CA (US)

(21) Appl. No.: 17/203,120

(22) Filed: Mar. 16, 2021

(51) Int. Cl. G16H 20/30; G16H 10/60; G16H 50/50; G16H 50/70; G16H 50/20; G06N 20/00; G06F 3/16; G06T 11/00; A63B 71/06; A63B 24/00

(52) U.S. Cl. CPC G16H 20/30; G16H 10/60; G16H 50/50; G16H 50/70; G16H 50/20; G06N 20/00; G06F 3/165; G06T 11/00; A63B 71/0622; A63B 24/0075; G16H 40/67

(57) ABSTRACT

Dynamically adaptable health experience based on data triggers is leveraged in a digital medium environment. For instance, a health manager system utilizes user-specific data such as health history data to generate audio content, interaction content, and exercise content for a health experience. Further, the health manager system monitors user state during a health experience and modifies the health experience in response to detecting various user states. A health entity interface is provided that enables various health entities to provide guidance for generating and modifying a health experience.

### BACKGROUND

A variety of different exercise systems are available that attempt to provide ways for individuals to engage in physical exercise. Examples of such systems include exercise equipment (e.g., treadmills, rowing machines, resistance machines), smart exercise monitors (e.g., wearable exercise monitoring devices), and exercise applications, e.g., for smartphones. Conventional exercise systems, however, suffer from a number of deficiencies. For instance, while some conventional systems provide output content in attempt to motivate users to engage in exercise, such content is typically drawn from a general collection of content (e.g., audio, video, etc.) and thus the content is not closely tailored to individual users. Further, conventional exercise systems typically have difficulty detecting subtle user and environmental changes and thus fail to provide exercise experiences that adapt to current user needs. This often results in users quitting their exercise routines or failing to engage in exercise altogether. Further, users that wish to participate in interactive exercise experiences are typically forced to manually locate and identify interactive content such as audio (e.g., music), video, images, and so forth. Thus, interactive exercise techniques provided by conventional exercise systems are burdensome on user and system resources required to generate custom exercise experiences (e.g., user time, memory, processor, and network bandwidth, etc.), require manual interaction with the systems, and/or do not achieve acceptable exercise experiences.

### SUMMARY

Dynamically adaptable health experience based on data triggers is leveraged in a digital medium environment. For instance, a health manager system utilizes user-specific data such as health history data to generate audio content, interaction content, and exercise content for a health experience. Further, the health manager system monitors user state during a health experience and modifies the health experience in response to detecting various user states. A health entity interface is provided that enables various health entities to provide guidance for generating and modifying a health experience.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures. Entities represented in the figures are indicative of one or more entities and thus reference is made interchangeably to single or plural forms of the entities in the discussion.

FIG. 1 is an illustration of an environment in an example implementation that is operable to employ techniques described herein.

FIG. 2 depicts an example system for generating a health experience according to the techniques described herein.

FIG. 3 depicts an example implementation of an exercise experience.

FIG. 4 depicts an example system for generating an avatar.

FIG. 5 depicts an example system for tracking user health progress via avatar modification.

FIG. 6 depicts a system 600 for configuring audio content for a health experience.

FIG. 7 depicts an example system for enabling health guidance from a health entity as part of a health experience.

FIG. 8 depicts an example system for providing interaction content as part of a health experience.

FIG. 9 is a flow chart depicting an example procedure for utilizing a user avatar as part of a health experience.

FIG. 10 is a flow chart depicting an example procedure for utilizing an updated user avatar as part of a health experience.

FIG. 11 is a flow chart depicting an example procedure for aggregating audio content for a health experience.

FIG. 12 is a flow chart depicting an example procedure for utilizing machine learning for audio content of a health experience.

FIG. 13 is a flow chart depicting an example procedure for aggregating interaction content for a health experience.

FIG. 14 is a flow chart depicting an example procedure for utilizing machine learning for interaction content of a health experience.

FIG. 15 is a flow chart depicting an example procedure for generating health instructions for a health experience.

FIG. 16 illustrates an example system including various components of an example device that are implementable as any type of computing device as described and/or utilized with reference to FIGS. 1-15 to implement aspects of the techniques described herein.

### DETAILED DESCRIPTION

Overview

To overcome the challenges to generating exercise routines presented in conventional exercise systems, dynamically adaptable health experience based on data triggers is leveraged in a digital medium environment. For instance, to mitigate the challenges of excessive burden on system resources experienced when attempting to obtain suitable exercise guidance content using conventional exercise systems, the described health manager system implements health experience generation techniques that reduce resource usage (e.g., memory and processor usage) in comparison with conventional exercise techniques, while providing highly interactive and adaptable content as part of a health experience. Further, the described techniques are able to implement machine learning aspects to provide quick and accurate content aggregation for a digital health experience.

Consider, for example, an implementation in which a user initiates a process for generating a health experience to assist the user in achieving a health goal, such as weight loss, muscle gain, improved cardiovascular health, pain reduction, and so forth. Accordingly, the user invokes the health manager system to initiate a health experience creation process for aggregating data into a set of health experience data for output to the user. As part of the creation process, for instance, the health manager system captures a digital image of the user and generates an original avatar based on the digital image. The original avatar, for example, represents a digital visual representation of the user and reflects various physical attributes of the user such as body mass, body dimensions, height to body mass ratio, and so forth. The original avatar is presented to the user and the user provides input to visually manipulate the original avatar to generate a target avatar. The user, for example, manipulates visual attributes of the original avatar to generate the target avatar to specify the user's health goal such as to mimic the user's desired physical appearance.

Accordingly, the health manager system compares the target avatar to the original avatar and generates an exercise set that is targeted to enable the user to obtain a physical appearance similar to the target avatar. The health manager system, for instance, determines a visual difference between the target avatar and the original avatar and correlates this difference to a change in physical attributes of the user. In at least one implementation this is performed by mapping portions of the avatars to corresponding regions of the user's body and determining differences between the body regions reflected in the visual difference between the target avatar and the original avatar. The health manager system then aggregates an exercise set targeted to enable the user to achieve the determined difference between the body regions. The health manager system, for example, includes exercise data that identifies exercises and indexes the exercises based on their respective health effects, e.g., weight loss, muscle mass gain (e.g., for specific body regions), increased flexibility, and so forth. Thus, the health manager system queries the exercise data to identify exercises labeled for achieving the specified health goal for the user, e.g., difference in user body regions. The health manager system then incorporates the exercise set into health experience data for output to the user as part of a health experience.

Further to the described techniques, the health manager system aggregates audio content for inclusion as part of the health experience. The health manager system, for example, accesses an audio (e.g., music) source for the user, such as an audio storage location, a user profile for an audio download and/or streaming service, and so forth. The health manager system aggregates a master playlist from the audio source, such as based on user preferences in conjunction with historical health experiences. The health manager system, for instance, maintains health history data for the user which includes express and/or implied user preferences regarding audio content. Thus, the health manager system aggregates the master playlist based on the user preferences. To enable a tailored playlist to be generated for a health experience, the health manager system determines a health experience context for the health experience. Generally, a health experience context represents data that describes various attributes of a health experience, such as exercises included in the health experience, exercise parameters (e.g., number of repetitions, number of sets, etc.), duration of the health experience, and so forth. Accordingly, the health experience system correlates the health experience context to instances of audio content from the master playlist to generate tailored audio content for the health experience. Various attributes of audio content are utilized to correlate to the health experience context, such as audio tempo, genre, artist, and so forth. The health manager system then incorporates the tailored audio content into health experience data for output to the user as part of a health experience. Further, the tailored audio content is dynamically modifiable during output of the health experience, such as based on detecting user state and/or health experience state.

The health manager system also aggregates interaction content for inclusion in a health experience. Generally, interaction content refers to content (e.g., audio, visual content, etc.) that is targeted to encourage user performance during user engagement with a health experience. The interaction content, for example, includes audible words and phrases for output during a health experience, such as to motivate a user to complete exercises included as part of the health experience. Accordingly, the health manager system utilizes various data to aggregate interaction content, such as user health history data, health experience context, and so forth. For instance, user health history indicates user reactions to particular types of interaction content, such as positive and/or negative reactions. Accordingly, types of interaction content historically observed to elicit positive user reactions is selected. Further, health experience context such as exercise type, duration, tempo, and so forth, are utilized to select interaction content. For instance, attributes of interaction content are matched to health experience context to determine optimal interaction content for output during the health experience. The interaction content is also modifiable during output of the health experience, such as to encourage user performance in response to determining user attributes such as facial expression, exercise form, exercise pace, and so forth.

Techniques for dynamically adaptable health experience based on data triggers are also implementable to incorporate health guidance from a health entity such as a physical therapist, a doctor, an exercise professional (e.g., a personal trainer), and so forth. A health entity, for example, utilizes a user's health goals to generate health guidance for achieving those goals, such as physical rehabilitation, weight loss, strength gain, body sculpting, and so forth. The health manager system, for instance, includes a health interface module that enables a health entity to obtain health-related information about a user from the health manager system and to communicate health guidance to the health manager system. In at least one implementation, a health entity is implemented remotely from the health manager system and thus communication between the health entity and the health manager system is performed over a network, such as a wired and/or wireless data network. The health manager system utilizes health guidance data received from a health entity to generate health instructions for inclusion as part of a health experience, such as specific exercises, exercise parameters, dietary suggestions, and so forth. Further, the health instructions are dynamically modifiable, such as based on modified health guidance received by the health manager system from a health entity.

Accordingly, the described techniques provide a custom tailored exercise experience for a user that aggregates health experience content based on various data triggers such as user health history, user preferences, health experience context, health guidance from health entities, and so forth. Thus, system resources (e.g., memory and processor resources) utilized for generating a health experience are conserved in contrast with conventional exercise content generation techniques that often fail to provide suitable health content for specific users and thus require manual input and system resources to identify suitable health content, e.g., exercises and exercise parameters. In this way, computationally efficient generation of health experiences provided by the described techniques are leveraged to reduce resource inefficiency experienced in conventional exercise systems and thus increase system efficiency.

### Term Definitions

These term definitions are provided for purposes of example only and are not intended to be construed as limiting on the scope of the claims.

As used herein the term “health experience” refers to an aggregation of computer-executable instructions and digital content that is configured for output as part of playback of a health experience by a computing system. For instance, a health experience includes digital audio, digital graphics, content transition triggers, and so forth, that are utilized to output the health experience.

As used herein the term “avatar” refers to a digital visual representation of a user, such as generated by computer graphics techniques. An avatar, for instance, is generated by capturing a digital image of a user (e.g., via a digital camera) and converting the digital image into a digital simulation of the image. Generally, an avatar is used for various purposes, such as to provide a visual representation of a current visual appearance of a user, a target visual appearance of a user, and so forth.

As used herein the term “interaction content” refers to content that is output (e.g., by a health manager system) in conjunction with user participation in a health experience. Interaction content, for example, includes audio and/or visual content that is output by a health manager system and that is targeted to elicit a particular user response such as to improve user performance and/or user mood during participation in a health experience.

As used herein the term “health entity” refers to an entity that provides health guidance for use in configuring health experiences. Examples of a health entity include a physical therapist, a doctor, an exercise professional, a dietician, and so forth. Further, a health entity includes an associated computing system that enables the health entity to interface with a health manager system.

In the following discussion, an example environment is first described that employs the techniques described herein. Example systems and procedures are then described which are performable in the example environment as well as other environments. Performance of the example systems and procedures is not limited to the example environment and the example environment is not limited to performance of the example systems and procedures. Finally, an example system and device are described that are representative of one or more computing systems and/or devices that are able to implement the various techniques described herein.

Example Environment

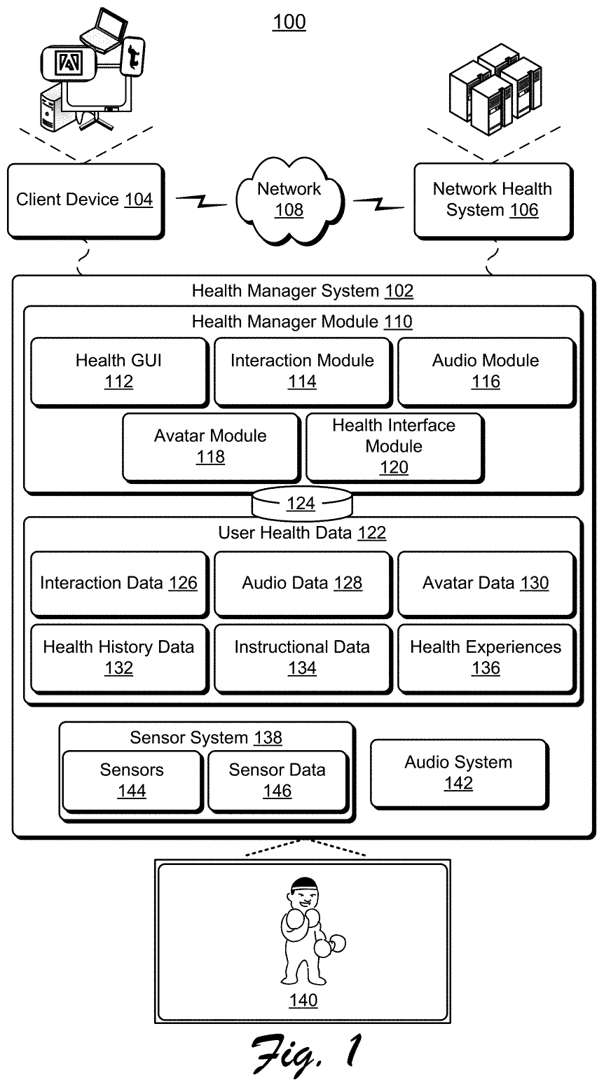

FIG. 1 is an illustration of an environment 100 in an example implementation that is operable to employ dynamically adaptable health experience based on data triggers as described herein. The illustrated environment 100 includes a health manager system 102 that is leveraged to implement techniques for dynamically adaptable health experience based on data triggers described herein. In this particular example, the health manager system 102 is implemented by a client device 104, a network health system 106, and/or via interaction between the client device 104 and the network health system 106. The client device 104 and the network health system 106, for example, are interconnected via a network 108 and thus are configured to communicate with one another to perform various aspects of dynamically adaptable health experience based on data triggers described herein. Generally, the network 108 represents a combination of wired and wireless networks and is implemented via any suitable architecture.

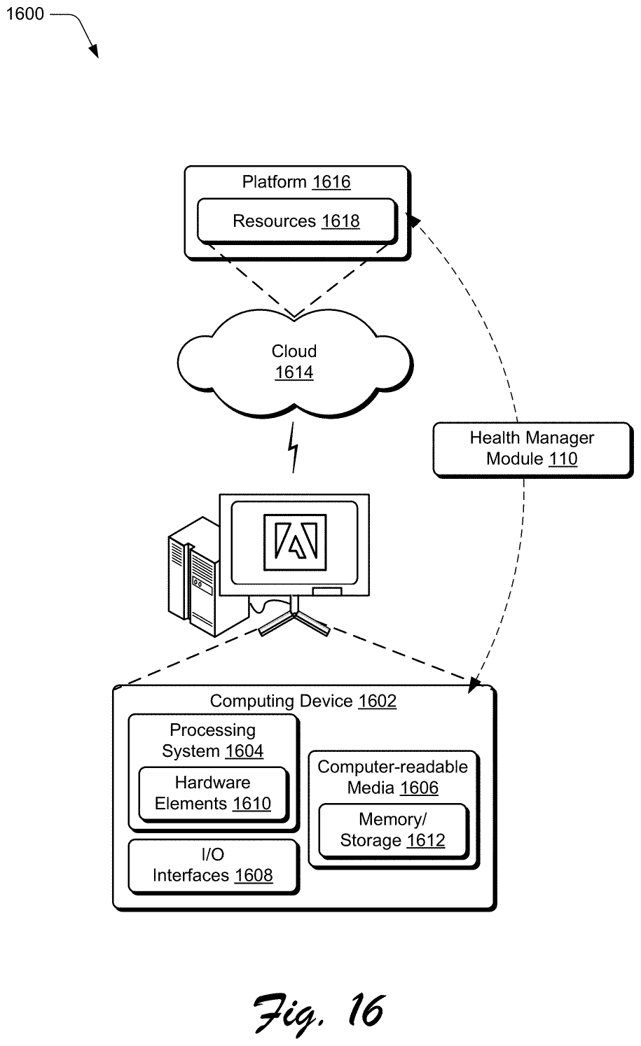

Examples of computing devices that are used to implement the client device 104 and the network health system 106 include a desktop computer, a laptop computer, a mobile device (e.g., assuming a handheld configuration such as a tablet or mobile phone), a server device, and so forth. Additionally, the network health system 106 is implementable using a plurality of different devices, such as multiple servers utilized by an enterprise to perform operations “over the cloud” as further described in relation to FIG. 16.

The health manager system 102 includes a health manager module 110 that is representative of functionality to provide tailored and adaptable exercise experiences. Accordingly, the health manager module 110 implements various functionality including a health graphical user interface (GUI) 112, an interaction module 114, an audio module 116, an avatar module 118, and a health interface module 120. Generally, the health GUI 112 represents functionality for receiving user interaction (e.g., via active input and/or passive input) to perform various exercise-related actions, as well as to output various exercise-related content. The interaction module 114 represents functionality to enable interactions between the health manager system 102 and a user. As further detailed below, for instance, the interaction module 114 monitors various user and/or environmental conditions and generates feedback such as in the form of motivational content based on the user/environmental conditions.

The audio module 116 represents functionality to identify, generate, and/or customize audio content for output to a user by the health manager system 102. For instance, the audio module 116 curates music and/or other audio content for output to specific users as part of exercise experiences. In at least one implementation, the audio module 116 obtains audio content from a user's personal playlist and arranges the audio content for output to a user, such as based on attributes of the audio content. The avatar module 118 represents functionality for generating and modifying user avatars. For instance, for a particular user, the avatar module 118 generates an avatar as a digital visual representation of the user. Further, the avatar is able to receive user interaction to specify different health goals and parameters, such as desired body shaping goals.

The health interface module 120 represents functionality for enabling different entities to interface with the health manager system 102, such as healthcare professionals, exercise professionals, and so forth. For instance, a particular user associated with the health manager system 102 has particular health goals and the health interface module 120 provides an interface via which another entity interacts with the health manager system 102 to assist in enabling the user to achieve those goals.

The health manager system 102 further includes user health data 122 stored on a storage 124. Generally, the user health data 122 includes data that is utilized by and results from operation of the health manager module 110. The user health data 122, for instance, includes interaction data 126, audio data 128, avatar data 130, health history data 132, instructional data 134, and health experiences 136. The interaction data 126 represents data that tracks user interactions with the health manager system 102 as well as output by the health manager system 102 pursuant to different exercise experiences. For instance, the interaction data 126 includes motivational content for output by the health manager system 102, e.g., audio content, video content, etc. Further, the interaction data 126 identifies user behaviors that occur in conjunction with output of the motivational content, e.g., user behaviors that coincide temporally with output of the motivational content.

The audio data 128 includes audio content (e.g., music, sound effects, etc.) as well as data describing user preferences for audio content, user behaviors that occur in conjunction with output of audio content, and so forth. In at least one implementation, the audio data 128 includes audio content that is obtained from a user's collection of audio content, such as downloaded from a storage location, streamed from a music streaming service based on the user's profile, and so forth. The avatar data 130 includes data that describes avatars generated for users as well as user interactions with avatars and modifications to avatars that occur based on morphological changes to users that are detected over time. The avatar module 118, for instance, generates an avatar for a user and stores the avatar in the avatar data 130, and updates the avatar in response to different events such as changes to a user's body that are detected.

The health history data 132 includes data that describes various health attributes of users, such as user health status at particular points in time (e.g., weight, height, body mass index (BMI), flexibility, strength, endurance, etc.), changes in user health status over time, user health milestones (e.g., flexibility goals, exercise goals, weight change goals, etc.), and so forth. The instructional data 134 includes data that is used to provide health-related instructions to users, such as instructions for exercise, physical therapy, diet, psychological recommendations, and so forth. For instance, a health entity such as a healthcare professional interacts with the health manager system 102 via the health interface module 120 to provide various health instructions that are stored in the instructional data 134. The health instructions are then output by the health manager system 102 to provide health instruction to a user as part of a user health session, such as an exercise routine, a physical therapy session, a health psychology session, and so forth.

The health experiences 136 include data aggregated from various sources and for output by the health manager system 102 as part of a health experience. For instance, the health manager module 110 selects instances of the audio data 128, the avatar data 130, the health history data 132, and the instructional data 134 and aggregates the data into different instances of health experiences 136. In at least some implementations, instances of the health experiences 136 include exercise information that describes different instances of exercises that are correlated to particular users, such as based on user health goals, health history data 132, instructional data 134, and so forth. Thus, instances of the health experiences 136 are output (e.g., via audio and/or video output) by the health manager system 102 to provide various types of health experiences such as exercise sessions, physical therapy sessions, and so forth.

The health manager system 102 further includes a sensor system 138, a display device 140, and an audio system 142. Generally, the sensor system 138 is representative of functionality to detect various physical and/or logical phenomena in relation to the health manager system 102, such as motion, light, image detection and recognition, time and date, position, location, touch detection, temperature, and so forth. To enable the sensor system 138 to detect such phenomena, the sensor system 138 includes sensors 144 that are configured to generate sensor data 146. Examples of the sensors 144 include hardware and/or logical sensors such as an accelerometer, a gyroscope, a camera, a microphone, a clock, biometric sensors, touch input sensors, position sensors, environmental sensors (e.g., for temperature, pressure, humidity, and so on), a scale for measuring user weight, a blood pressure sensor, geographical location information sensors (e.g., Global Positioning System (GPS) functionality), and so forth. In at least some implementations, the sensor data 146 represents raw sensor data collected by the sensors 144. Alternatively or in addition, the sensor data 146 represents raw sensor data from the sensors 144 that is processed to generate processed sensor data, such as sensor data from multiple sensors 144 that is combined to provide more complex representations of user and/or environmental state than is provided by a single sensor 144. Generally, the sensor data 146 is usable for various purposes, such as capturing user physical and health attributes for enabling different functionality of the health manager system 102.

The display device 140 represents functionality for visual output of various aspects of techniques for dynamically adaptable health experience based on data triggers. The display device 140, for instance, outputs the health GUI 112, and is operable to receive user interaction to perform various aspects of the described techniques. A user, for example, provides input to the health GUI 112 to invoke the health manager module 110. Additionally, functionality of the health manager module 110 is invocable by other entities such as based on interaction with the health interface module 120. The audio system 142 represents functionality for output of audible content by the health manager system 102, such as audio data 128 as part of a user health experience. The health manager system 102, for instance, utilizes the display device 140 and the audio system 142 to output video and audio output for the health experiences 136.

Having considered an example environment and system, consider now a discussion of some example details of the techniques for dynamically adaptable health experience based on data triggers in a digital medium environment in accordance with one or more implementations.

Implementation Details

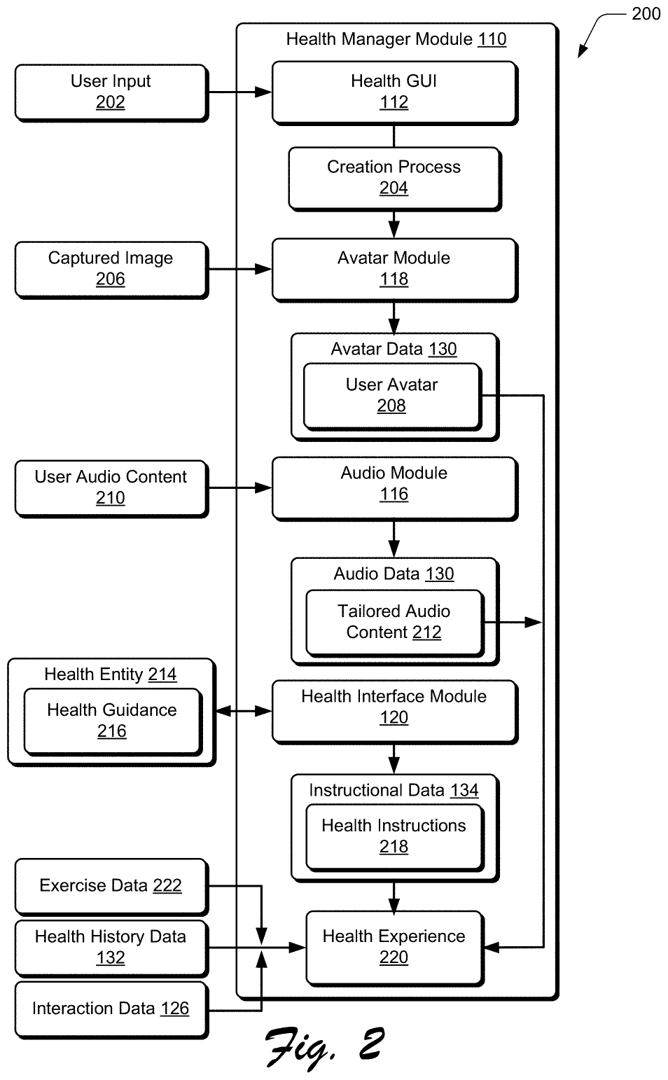

FIGS. 2-8 depict different implementation details for dynamically adaptable health experience based on data triggers in accordance with one or more implementations. For instance, FIG. 2 depicts an example system 200 for generating a health experience according to the techniques described herein. The system 200, for example, describes an overview of generating a health experience via the health manager module 110. Generally, the various modules discussed herein are implementable in hardware, firmware, software, and/or combinations thereof.

In the system 200, the health manager module 110 receives user input 202 to the health GUI 112 to invoke functionality of the health manager module 110. A user, for instance, interacts with the health GUI 112 to initiate a creation process 204 for creating a health experience for the user. The creation process 204, for example, implements a set of subprocesses by the various modules of the health manager module 110 for generating a health experience. Generally, the user input 202 represents various types of input, such as touch input to the display device 140, keyboard input, mouse/cursor input, voice input, and so forth.

Accordingly, the creation process 204 invokes the avatar module 118 to obtain a captured image 206 of the user. The avatar module 118, for example, invokes the sensor system 138 to obtain the captured image 206 from the sensors 144, e.g., a camera. Generally, the captured image 206 represents a digital image of the user. Utilizing the captured image 206, the avatar module 118 generates a user avatar 208 that represents a digital representation of the user generated from the captured image 206. The avatar module 118, for example, generates the user avatar 208 as a digital graphical simulation of the captured image 206, such as a 2-dimensional and/or 3-dimensional representation of the user. Further, the avatar 208 reflects morphological features of the user from the captured image 206, such as body shape and dimensions. As further detailed below, the user avatar 208 is usable to enable the user to specify various exercise goals as well as to track user progress.

The creation process 204 further invokes the audio module 116 to obtain user audio content 210 and to generate tailored audio content 212 using the user audio content 210. Generally, the user audio content 210 is obtainable in various ways, such as from an audio storage location associated with the user, from a user profile with an audio service (e.g., an audio download and/or streaming service), user selection of instances of audio content, and so forth. The audio module 116 generates the tailored audio content 212 based on various criteria, such as user preferences, observed user behaviors, attributes of exercises to be included as part of an exercise experience, and so forth. Further details concerning generating the tailored audio content 212 are discussed below. Accordingly, the audio module 116 stores the tailored audio content 212 as part of the audio data 128.

Further to the system 200, a health entity 214 interacts with the health interface module 120 to specify health guidance 216. The health guidance 216, for instance, represents types, descriptions, parameters, recommendations, and so forth, for exercises to be included in a health experience. Generally, the health entity 214 represents an entity engaged in providing health services and/or health recommendations, such as a physician, physical therapist, chiropractor, exercise trainer, dietician, and so forth. The health entity 214, for instance, represents a human that provides the health guidance 216, a logical entity that generates the health guidance 216 (e.g., a machine learning model), and/or combinations thereof. In at least one implementation, the health manager module 110 utilizes the health interface module 120 to present a menu of exercises to the health entity 214 and the health entity selects a set of exercises from the menu to generate the health guidance 216. The health interface module 120 utilizes the health guidance 216 to generate health instructions 218 for use in generating a health experience. The health interface module 120, for instance, converts the health guidance 216 into the health instructions 218 that are able to be output by the health manager system 102.

Accordingly, the health manager module 110 generates a health experience 220 based on the creation process 204. Further, in addition to the previously described processes, the creation process 204 utilizes exercise data 222, the health history data 132, and the interaction data 126 for generating the health experience 220. The exercise data 222 generally represents different exercises that are available for generating the health experience 220 and are obtainable from various sources, such as the health manager system 102, the network health system 106, and so forth. The creation process 204 utilizes the health history data 132 for various purposes, such as to select exercises from the exercise data 222, to specify parameters for exercises (e.g., repetitions, sets, form, etc.), to specify a time duration for the health experience 220, and so forth. Further, the interaction data 126 is utilized to generate interaction content for the health experience 220, such as motivational words and phrases to be output as part of the health experience 220.

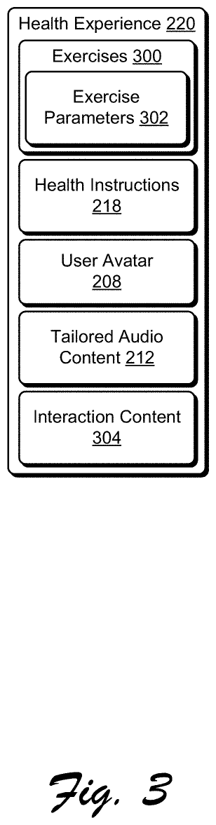

FIG. 3 depicts an example implementation of the exercise experience 220. The exercise experience 220 includes exercises 300 with exercise parameters 302, the health instructions 218, the user avatar 208, the tailored audio content 212, and interaction content 304. The exercises 300, for instance, are obtained from the exercise data 222. Further, the exercise parameters 302 represent suggested user parameters for performing the exercises 300, such as a number of repetitions, a number of sets, pace information, and so forth, for each exercise 300. In at least one implementation, the exercises 300 and the exercise parameters 302 are generated based on the health history data 132. For instance, past user performance for instances of the exercises 300 (e.g., repetitions, sets, user form, etc.) is identified in the health history data 132, and the health manager module 110 utilizes this data to select the exercises 300 and to specify the exercise parameters 302.

As introduced above, the health instructions 218 are generated based on health guidance 216 from the health entity 214, and generally represent user guidance for participating in the health experience 220. In at least one implementation, the exercises 300 and/or the exercise parameters 302 are generated based on the health instructions 218. The user avatar 208 is utilized to provide a visual representation of a user engaging with the health experience 220. In at least one implementation, user progress over time is reflected by the user avatar 208, such as user progression toward a specified health goal, e.g., weight loss. The tailored audio content 212 is output as part of the health experience 220 and as discussed below, is further customizable based on various context information pertaining to output of the health experience 220. Further, interaction content 304 is extracted from the interaction data 126 and is output as part of the health experience 220. The interaction content 304 is also modifiable during output of the health experience 220 such as based on detected changes in user state, environmental state, and so forth. Thus, the health experience 220 provides a custom tailored and dynamically modifiable set of health-related data for output as part of a health-related session.

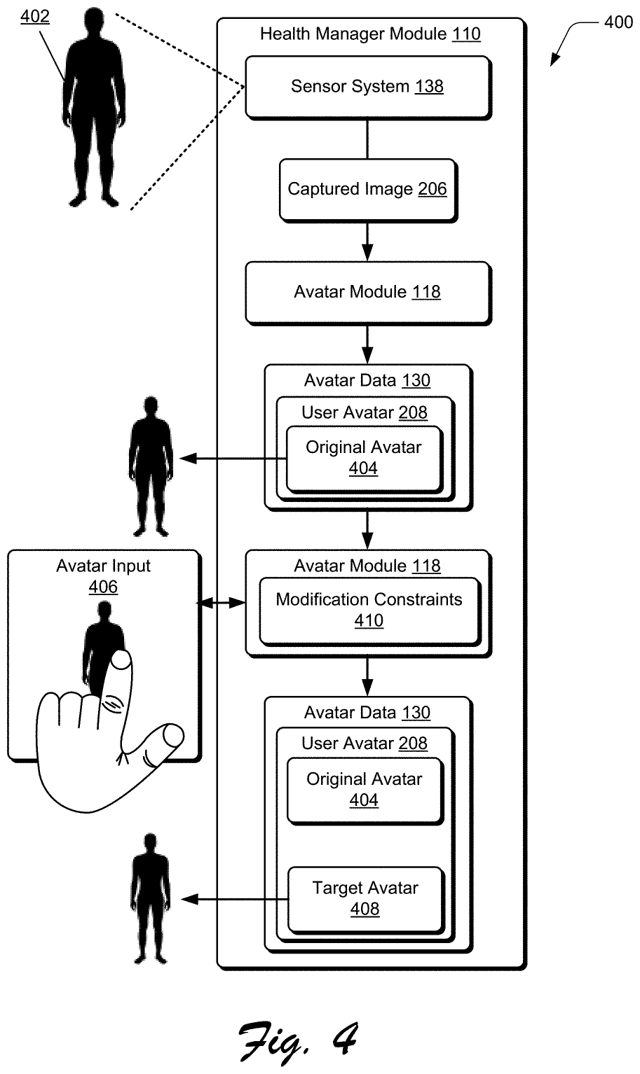

FIG. 4 depicts an example system 400 for generating an avatar. The system 400, for example, provides further detail for portions of the system 200, including generating the user avatar 208 as part of the creation process 204. In the system 400 the sensor system 138 captures the captured image 206 of a user 402, such as via a camera and/or other image capture device. The avatar module 118 processes the captured image 206 to generate an original avatar 404 of the user avatar 208. In at least one implementation, the original avatar 404 represents a physical appearance of the user 402 at a particular period in time, such as when the user 402 initially registers (e.g. creates a profile) with the health manager system 102. Alternatively or additionally, the original avatar 404 represents the user 402 when the user begins a particular health program, such as a physical fitness routine.

The user 402 then interacts with the health manager module 110 to provide avatar input 406 to the original avatar 404. The avatar input 406, for instance, represents input to modify an appearance of the original avatar 404, such as via touch input, mouse/cursor input, etc. For example, the user 402 utilizes the avatar input 406 to modify a shape of the original avatar 404 to indicate a health goal of the user 402, such as weight reduction, increase in muscle mass, and so forth. Accordingly, based on the avatar input 406, the avatar module 118 generates a target avatar 408 that represents the original avatar 404 as modified by the avatar input 406. The target avatar 408, for instance, reflects a target visual appearance of the user 402, such as a health goal that the user 402 sets via the avatar input 406. In at least one implementation, the avatar module 118 enforces a set of modification constraints 410 to constrain (e.g., limit) allowed modification of the original avatar 404 by the avatar input 406. For instance, the modification constraints 410 correlate to certain physical attributes of the user 402 that are likely not physically modifiable via the health experience 220, such as user height, body type (e.g., ectomorph, endomorph, mesomorph), limb length, and so forth. Thus, the avatar input 406 is prevented from modifying the original avatar 404 in a way that violates a modification constraint 410.

Accordingly, the target avatar 408 is utilized as part of generating the health experience 220. For instance, the health manager module 110 compares the original avatar 404 to the target avatar 408 and selects the exercises 300 and the exercise parameters 302 that are most likely to enable the user 402 to achieve a physical appearance similar to the target avatar 408. Alternatively or additionally, the original avatar 404 and the target avatar 408 are provided to the health entity 214 via the health interface module 120 and the health entity 214 generates the health guidance 216 as recommendations for the user 402 to achieve a physical appearance similar to the target avatar 408.

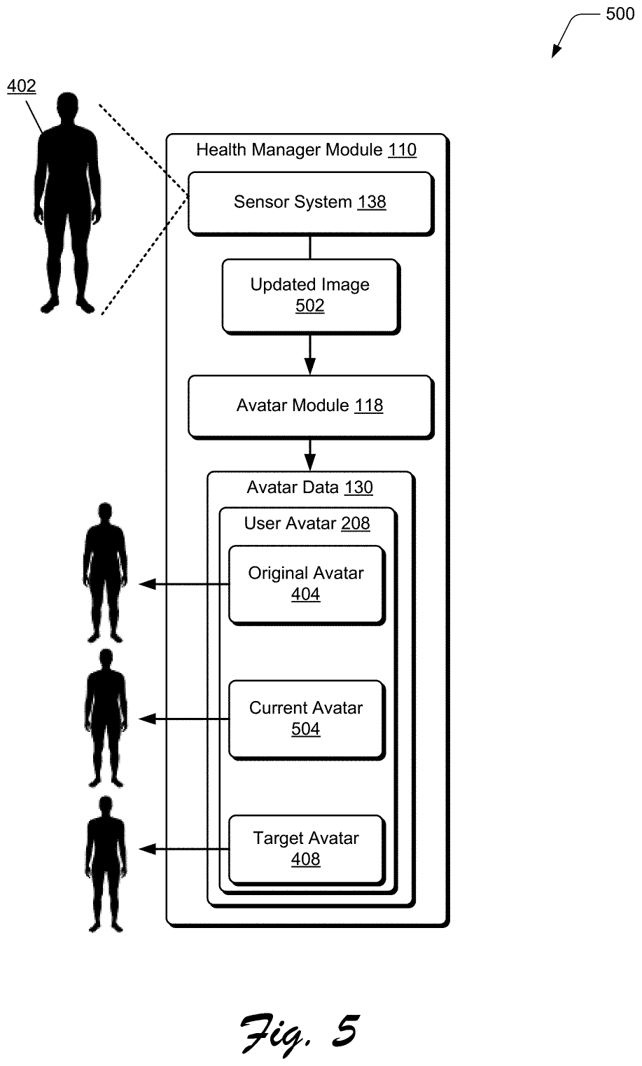

FIG. 5 depicts an example system 500 for tracking user health progress via avatar modification. The system 500, for instance, is implemented as an extension of the systems described above. In the system 500, the sensor system 138 captures an updated image 502 of the user 402. The updated image 502, for instance, is captured at a subsequent date from which the captured image 206 was captured, such as weeks or months after the captured image 206 was obtained. The user 402, for instance, engages in the health experience 220 and/or other health experiences over a period of time after the captured image 206 was captured and the updated image 502 is captured after this period of time.

Accordingly, the avatar module 118 utilizes the updated image 502 to generate a current avatar 504 that represents a digital representation of the user 402 at the point in time that the updated image 502 was captured. The current avatar 504, for example, reflects morphological features of the user from the updated image 502, such as body shape and dimensions. In at least one implementation, the health manager module 110 displays the current avatar 504, the original avatar 404, and the target avatar 408, such as via the health GUI 112. Generally, this provides a visual indication of progress of the user 402 toward a health goal. Further, the current avatar 504 is usable to modify the health experience 220, such as to update the exercises 300 and/or the exercise parameters 302. For instance, the health manager module 110 compares the current avatar 504 to the original avatar 404 to determine if progress is made toward a health goal indicated by the target avatar 408, and if so, how much progress. Progress, for instance, is indicated by body mass reduction and/or increased muscle mass. If little or no progress is observed, for instance, the health manager module 110 modifies the exercises 300 and/or the exercise parameters 302, such as by adding and/or replacing current exercises 300, adding additional repetitions and/or sets to the exercise parameters, and so forth. Additionally or alternatively, the current avatar 504 is provided to the health entity 214 and the health entity 214 provides guidance for achieving a health goal based on the current avatar 504. The health entity 214, for instance, compares the current avatar 504 to the original avatar 404 and the target avatar 408 to gauge progress of the user 402 toward a health goal. The health entity 214 then provides guidance to the user 402 via the health interface module 120, such as via an update to the health guidance 216.

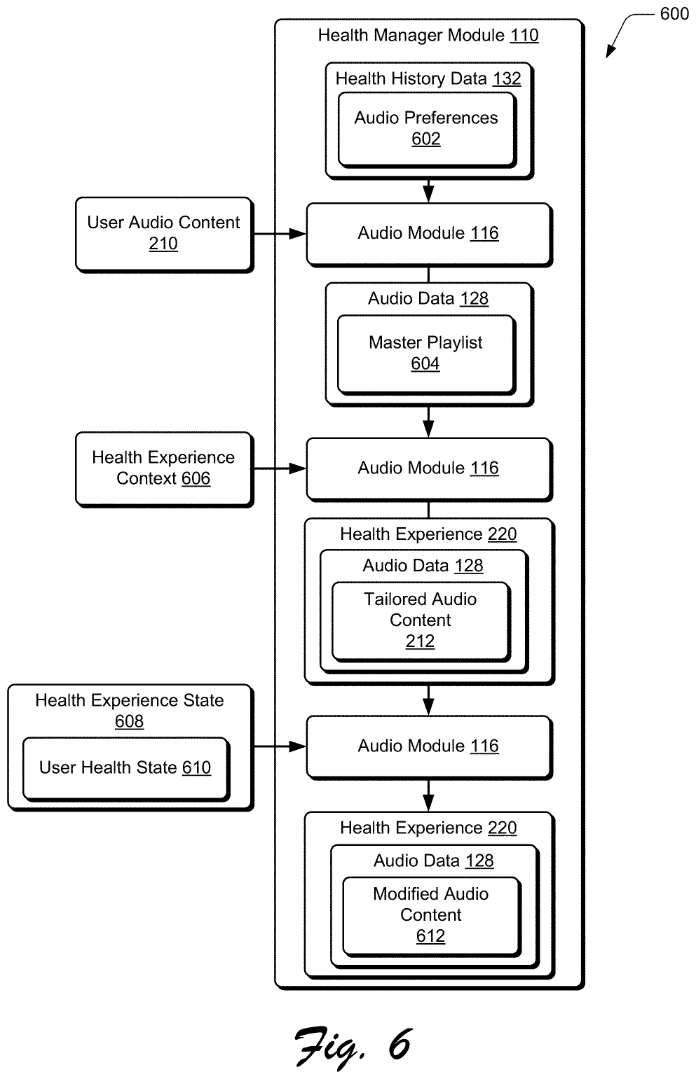

FIG. 6 depicts a system 600 for configuring audio content for a health experience, such as the health experience 220. In the system 600, the audio module 116 determines audio preferences 602, such as for the user 402. Generally, the audio preferences 602 are determinable in various ways, such as based on preferences indicated in the health history data 132, user input to identify audio preferences, based on preferences obtained from an external source such as an audio service, and so forth. The audio preferences 602 include various types of audio preferences, such as based on genre, artist, songs, audio attributes (e.g., audio composition attributes), and so forth. Further, the audio module 116 accesses the user audio content 210 and applies the audio preferences 602 to the user audio content 210 to generate a master playlist 604. Instances of audio content from the user audio content 210, for instance, are matched to the audio preferences 602 to aggregate the master playlist 604 of audio content.

Further to the system 600, the audio module 116 determines health experience context 606 and processes the master playlist 604 based on the health experience context 606 to generate tailored audio content 212. The health experience context 606 represents data that describes various attributes pertaining to a health experience (e.g., the health experience 220), such as the exercises 300 and exercise parameters 302, user health history, time of day and/or day of week that a health experience is output and/or scheduled to be output, and so forth. For instance, consider that the exercises 300 and/or the exercise parameters 302 include a fast-paced exercise portion for the health experience 220. The audio module 116 detects the fast paced portion and includes up-tempo audio from the master playlist 604 in the tailored audio content 212. Accordingly, the tailored audio content 212 is indicated for playback as part of the health experience 220 and/or other health experiences.

Further to the system 600, after generating the tailored audio content 212, the audio module 116 receives health experience state 608 data, such as from the sensor system 138. Generally, the health experience state 608 represents data received in conjunction with output of the health experience 220, such as during output of the health experience 220 and associated playback of the tailored audio content 212. In at least one implementation, the health experience state 608 includes indications of user health state 610, such as facial expression, posture, spoken words and phrases, mood and/or sentiment information (e.g., determined from facial expression and/or spoken words), heart rate, breath rate, and so forth.

In at least one implementation, the user health state 610 is determined by detecting muscular pain and fatigue in correlation with facial analysis. For instance, the health manager module 110 utilizes the Facial Action Coding System (FACS) to monitor user facial expressions and imply user health state 610 based on the facial expressions. As one example, a pull up of action unit 12 (AU12) indicates a positive emotion while a pull down indicates pain and fatigue. Facial expression is also combinable with speed of user movement and current status of exercise repetitions to provide motivation in the form of contextual music recommendation, music volume increase and/or verbal motivation, e.g., via interaction content.

Alternatively or in addition, the health experience state 608 includes environment state data, such as ambient sounds detected in a local environment, temperature, light level, and so on. Based on the health experience state 608 the audio module 116 modifies the tailored audio content 212 to generate modified audio content 612. The modified audio content 612, for instance, represents a rearrangement of audio content from the tailored audio content 212 and/or a supplementation and/or replacement of audio content 212 with audio content from the master playlist 604. Additionally or alternatively, the modified audio content 612 modifies audio attributes of content of tailored audio content 212, such as output volume, tempo, tonal attributes, etc.

Consider, for example, that the health experience state 608 indicates that the user 402 is in a poor mood, such as based on facial expression, detected speech, and so forth. Accordingly, the audio module 116 generates the modified audio content 612 to include audio targeted to improve user mood, such as more upbeat audio content than is currently specified by the tailored audio content 212. As another example, the health experience state 608 indicates that the user 402 is exhibiting a slowing pace of movement and/or poor exercise form, and thus the audio module 116 includes more upbeat audio content in the modified audio content 612 to attempt to motive the user to improve their pace/form. As yet another example, the health experience state 608 indicates that audio output of the tailored audio content 212 is causing echo within the local environment and thus the audio module 116 reduces output volume of the modified audio content 612, or the health experience state 608 indicates high ambient noise levels (e.g., above a threshold decibel level) and thus the audio module 116 increases output volume of the modified audio content 612. These examples of the health experience state 608 and audio content modification are presented for purpose of example only, and it is to be appreciated that a wide variety of different types of state information and audio modifications are able to be implemented in accordance with the implementations described herein.

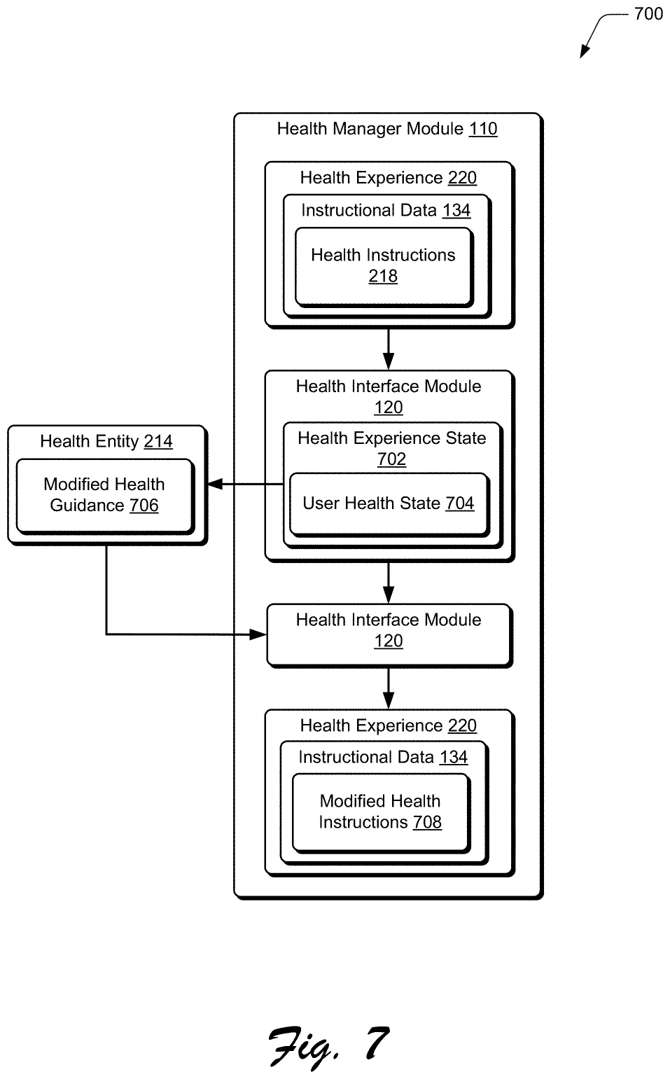

FIG. 7 depicts an example system 700 for enabling health guidance from a health entity as part of a health experience. The system 700, for example, is implemented in conjunction with the systems described above. In the system 700, the health experience 220 includes the health instructions 218 generated based on the health guidance 216 from the health entity 214, such as discussed above with reference to the system 200. Further, the health manager module 110 determines a user health state 704 based on various user state information such as user weight, BMI, heart rate, blood pressure, and so forth. In at least one implementation, the health manager module 110 leverages the sensor system 138 to capture the user health state 704. The health manager module 110 also determines health experience state 702, examples of which are discussed above with reference to the health experience state 608. The health experience state 702, for instance, includes a user health state 704 detected in conjunction with participation in the health experience 220, such as user reaction to the health experience 220 detected via facial expression, verbal output, posture, and so forth. Additionally or alternatively, the health experience state 702 includes state information for the health experience 220 itself, such as identifiers for the exercises 300 and/or the exercise parameters 302, the health instructions 218, and so forth.

Accordingly, the health experience state 702 is communicated to the health entity 214, such as via a push and/or pull data communication between the health interface module 120 and the health entity 214. The health entity 214 utilizes the health experience state 702 including the user health state 704 to generate modified health guidance 706. The health entity 214, for instance, determines based on the user health state 704 that the health instructions 218 are to be modified to accommodate a change in user health status indicated by the user health state 704. In at least one implementation, the health entity 214 compares the user health state 704 to the health history data 132 for the user to identify a change in user health status indicated by the user health state 704. Accordingly, the health entity 214 generates the modified health guidance 706 based on the user health state 704, e.g., in response to a change in user health status.

In an optional implementation the health entity 214 utilizes the health experience state 702 to generate the modified health guidance 706. For instance, the modified health guidance 706 suggests modification to the exercises 300 and/or the exercise parameters 302 based on the user health state 704 and/or the health experience state 702. Consider, for example, an implementation where the user health state 704 indicates that the user has gained weight, e.g., since user health state was previously determined by the health manager module 110. Accordingly, the modified health guidance 706 includes recommendations for stopping weight gain and/or losing weight, such as additional exercises and/or exercise repetitions, more frequent exercise, dietary changes, and so forth. Consider another example where the health experience state 702 indicates that the user appears to be overexerting themself during the health experience 220, such as based on detecting facial expression indicating pain and/or excessive fatigue, excessively high heart rate, verbal feedback from the user, and so forth. Accordingly, the health entity 214 generates the modified health guidance 706 to suggest changes to the health experience 220 to reduce physical exertion by the user, such as fewer and/or less rigorous exercises 300. As yet another example, the health experience state 702 indicates that the user is not exerting themself during the health experience, such as based on low heart rate, relaxed facial expression, etc. In this example, the health entity 214 generates the modified health guidance 706 to suggest additional exercises 300, additional exercise repetitions, additional weight resistance, and so forth, to attempt to increase user exertion as part of the heath experience 220.

Accordingly, the health entity 214 communicates the modified health guidance 706 to the health manager module 110 via the health interface module 120, and the health manager module 110 generates modified health instructions 708 based on the modified health guidance 706. The modified health instructions 708, for instance, represent changes to the health instructions 218, such as by adding, deleting, and/or modifying the health instructions 218 to generate the modified health instructions 708. The modified health instructions 708 indicate various changes to the health experience 220, such as changes to exercises (e.g., additional exercises and/or repetitions, fewer exercises and/or repetitions, changes to exercise form, etc.), suggested dietary changes, changes to motivational content, and so forth. Thus, the health experience 220 incorporates the modified health instructions 708 for output to the user.

In at least one implementation, the modified health guidance 706 and the modified health instructions 708 are generated asynchronously with output of the health experience 220, e.g., while the health experience 220 is not being output and/or the user is not engaged in the health experience 220. Alternatively or additionally, the modified health guidance 706 and the modified health instructions 708 are generated synchronously with output of the health experience 220, e.g., while the health experience 220 is being output and/or the user is engaged in the health experience 220. The system 700, for example, is implementable to dynamically modify the health experience 220 based on data collected during output of the health experience 220, e.g., the user health state 704 and/or the health experience state 702. The health entity 214, for instance, collects the user health state 704 and/or the health experience state 702 in real time while the user is detected as being engaged in the health experience 220, and generates the modified health guidance 706 and communicates the modified health guidance 706 to the health manager module 110 in real time. Generally, this enables the health manager module 110 to generate the modified health instructions 708 to dynamically adapt the health experience 220 in real time while the user is engaged with the health experience 220, such as to accommodate changes in user health state 704, changes in health experience state 702, and so forth.

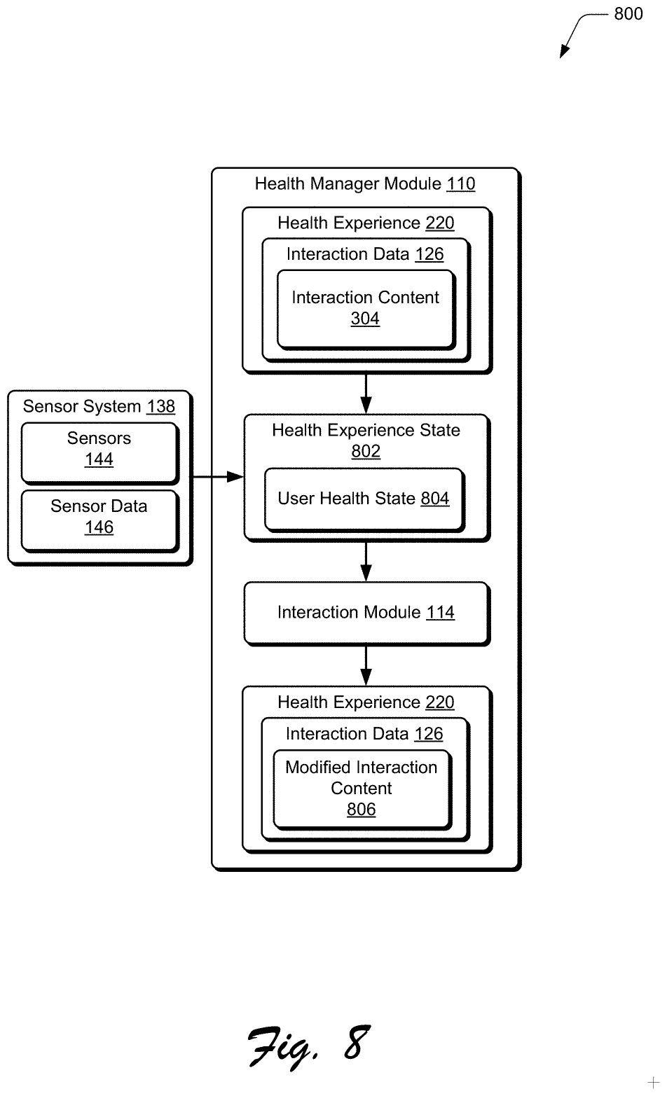

FIG. 8 depicts an example system 800 for providing interaction content as part of a health experience. The system 800, for example, is implemented in conjunction with the systems described above. In the system 800, the health experience 220 includes the interaction content 304, such as discussed above. The interaction content 304, for instance, represents content for output by the health manager module 110 as part of the health experience 220. The interaction content 304 includes various types of content generated to instruct and motivate a user in conjunction with the health experience 220, such as audible and visual content. For instance, the interaction content 304 includes words and/or phrases for user instruction and motivation as part of the health experience 220. In an instructional implementation, for example, the interaction content 304 includes instructions for performing the exercises 300, such as names and descriptions for the exercises 300 and the exercise parameters 302, instructions and suggestions for performing the exercises 300, instructions for transitioning between different exercises 300, and so forth. In a motivational scenario, the interaction content 304 includes words, phrases, and/or visual cues targeted to motivate the user during output of the health experience 220, such as encouraging words and phrases (e.g., “doing great, just 5 more,” “you can do it try pushing a bit more”), feedback regarding user exercise (e.g., “you're slowing down, try to pick up the pace,” “you're bending your back, try straightening up”), visual prompts for user encouragement and exercise correction, and so forth. Thus, the interaction content 304 is output for user motivation and instruction before, during, and/or after output of the health experience 220.

Further to the system 800 the interaction module 114 receives health experience state 802 data which includes user health state 804 data and utilizes the data to generate modified interaction content 806 for inclusion with the health experience 220. In at least one implementation, the user health state 804 and/or the health experience state 802 are based at least in part on sensor data 146 received from the sensor system 138. Examples of the user health state 804 and the health experience state 802 are presented above, such as with reference to the user health state 704 and the health experience state 702, respectively. Generally, the user health state 804 and the health experience state 802 are interpreted by the interaction module 114 as including state information indicating that the interaction content 304 is to be modified to generate the modified interaction content 806. For instance, consider an example wherein during user engagement with the health experience 220 the user health state 804 indicates that the user's heart rate is low, e.g., below a target heart rate zone. Accordingly, the interaction module 114 generates the modified interaction content 806 to include an instructional phrase and/or visual cue for the user to increase the pace of exercise to attempt to elevate their heart rate into the target zone. As another example, the user health state 804 indicates that the user is exhibiting signs of pain and/or exhaustion (e.g., based on facial expression) and thus the modified interaction content 806 instructs the user to slow their pace.

In at least one implementation, the health manager module 110 utilizes the sensor data 146 to perform skeletal tracking of the user in conjunction with the health experience 220, such as to identify form, speed (e.g., tempo), and smoothness during exercises by measuring movements of user skeletal points. Thus the system is able to utilize skeletal tracking and facial expression detection to detect user fatigue, such as based on slow and/or irregular movement and detected pain based on observing facial expression. This data is utilized to provide feedback (e.g., in real time) to enable the interaction module 114 to generate the interaction content 304 and the modified interaction content 806 to target user motivation in conjunction with the health experience 220.

As yet another example, the health experience state 802 indicates that the user is slowing down during a particular exercise set and the modified interaction content 806 includes an encouraging phrase such as “you've got this only 5 more.”

In at least one implementation, the modified interaction content 806 is generated synchronously with output of the health experience 220, e.g., while the health experience 220 is being output and/or the user is engaged in the health experience 220. The system 800, for example, is implementable to dynamically modify the health experience 220 based on data collected during output of the health experience 220, e.g., the user health state 804 and/or the health experience state 802. The interaction module 114, for instance, collects the user health state 804 and/or the health experience state 802 in real time while the user is detected as being engaged in the health experience 220, and generates the modified interaction content 806 for output as part of the health experience. Generally, this enables the health manager module 110 to generate the modified interaction content 806 to dynamically adapt the health experience 220 in real time while the user is engaged with the health experience 220, such as to accommodate changes in user health state 804, changes in health experience state 802, and so forth.

Alternatively or additionally, the modified interaction content 806 is generated asynchronously with output of the health experience 220, e.g., while the health experience 220 is not being output and/or the user is not engaged in the health experience 220. For instance, as part of initiating output of the health experience 220 (e.g., as part of calibrating the health experience 220), the health manager module 110 receives the user health state 804 and the health experience state 802 and generates the modified interaction content 806 to be used as part of subsequent output of the health experience 220. Generally, this enables the health experience 220 to be adapted to a current user health state 804 prior to the user engaging in the health experience 220.

As an alternative or additional implementation, the health manager module 110 collects the user health state 804 after output of the health experience 220, e.g., within 5 minutes after the user is finished engaging with the health experience 220. The interaction module 114 then utilizes the user health state 804 and the health experience state 802 to generate the modified interaction content 806 for use as part of the health experience 220 at a later time. Generally, this enables the user health state 804 to be utilized by the health manager module 110 as feedback for adapting future output of the health experience 220, e.g., to adapt to changes in user health, environmental conditions, and so forth.

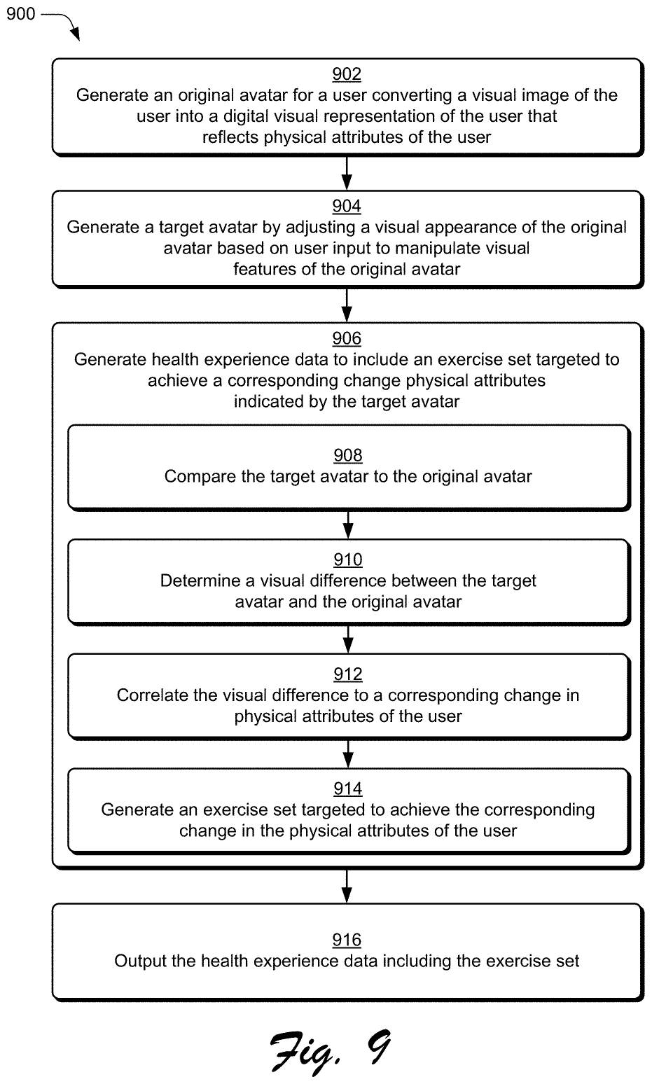

Having discussed some implementation details, consider now some example methods for dynamically adaptable health experience based on data triggers. FIG. 9, for instance, depicts an example method 900 for utilizing a user avatar as part of a health experience. Step 902 generates an original avatar for a user converting a visual image of the user into a digital visual representation of the user that reflects physical attributes of the user. The avatar module 118, for instance, obtains a digital image of a user, such as from the sensor system 138. The avatar module 118 then converts the digital image into an artificial digital visual representation of the user (an avatar) that reflects physical attributes of the user, e.g., physical dimensions of the user such as girth relative to height, body outline appearance, waist size, etc.

Step 904 generates a target avatar by adjusting a visual appearance of the original avatar based on user input to manipulate visual features of the original avatar. For instance, the avatar module 118 receives user input to manipulate visual features of the original avatar and adjusts a visual appearance of the original avatar based on the manipulated visual features. The user input, for example, manipulates visual features such as to reduce waist size, stomach size, increase muscle mass in various regions of the original avatar, and so forth. For instance, the original avatar includes a representation of a physical dimension of the user, and a particular manipulated visual feature involves a manipulation of the representation of the physical dimension of the user to generate the target avatar.

Step 906 generates health experience data to include an exercise set targeted to achieve a corresponding change physical attributes indicated by the target avatar. The health manager module 110, for instance, generates a set of exercises that are targeted to enable a physical appearance of the user to resemble a visual appearance of the target avatar. In at least one implementation, the health manager module 110 generates the health experience data by:

Step 908 compares the target avatar to the original avatar. For example, the health manager module 110 compares a visual appearance of the target avatar to a visual appearance of the original avatar. Step 910 determines a visual difference between the target avatar and the original avatar. The health manager module 110, for example, determines visual differences between the target avatar and the original avatar, such differences in surface area, width, limb mass, and so forth. Step 912 correlates the visual difference to a corresponding change in physical attributes of the user. In at least one implementation, the avatar data 130 includes mappings of avatar regions to corresponding physical regions of a user, e.g., avatar waist region correlates to user waist, avatar stomach region correlates to user stomach, avatar shoulder region correlates to user shoulder, and so forth. Accordingly, the health manager module 110 utilizes this mapping to correlate visual changes indicated by the target avatar to corresponding changes to physical attributes of the user.

Step 914 generates the health experience data to include an exercise set targeted to achieve the corresponding change in the physical attributes of the user. The exercise data 222, for example, identifies exercises that are targeted to achieve certain health goals, such as weight loss, muscle mass gain (e.g., for specific body regions), physical strength, flexibility, pain reduction, and so forth. Thus, the health manager module 110 maps the change in physical attributes to a particular exercise and/or exercises identified as being targeted to achieve the change in physical attributes. The health manager module 110 includes the exercise set as part of an overall health experience, e.g., the exercises 300 of the health experience 220.

Step 916 outputs the health experience data including the exercise set. The health manager module 110, for example, outputs the health experience 220 including the exercises 300 and various other attributes of the health experience 220. In at least one implementation, the health manager module 110 outputs visual attributes of the health experience 220 via the health GUI 112 displayed on the display device 140, and outputs audio attributes of the exercise experience 220 via the audio system 142.

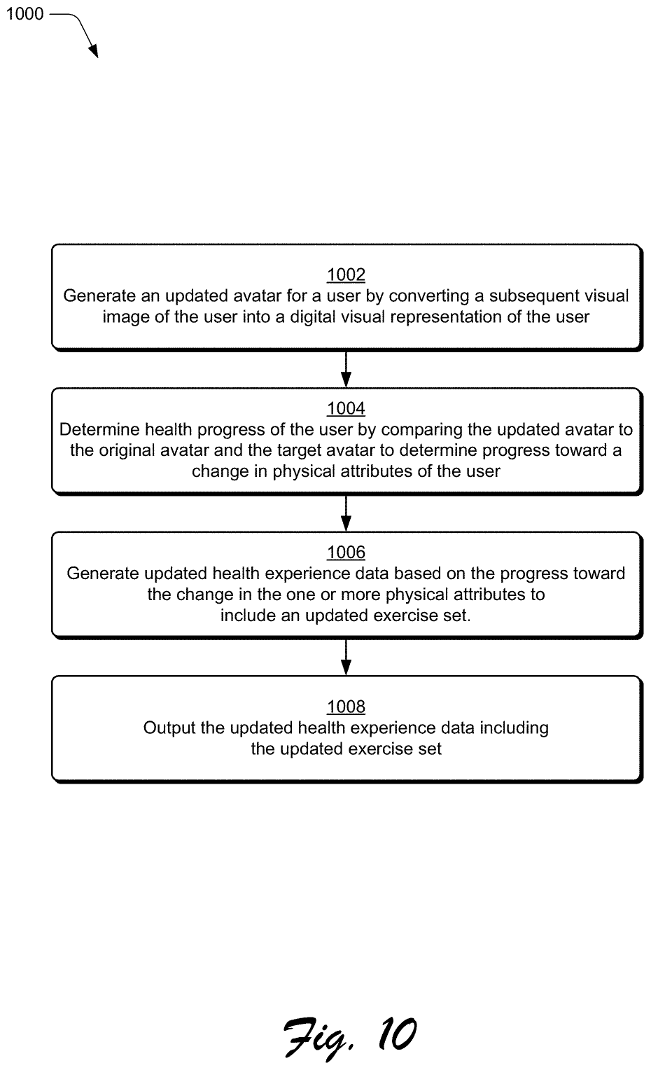

FIG. 10 depicts an example method 1000 for utilizing an updated user avatar as part of a health experience. The method 1000, for example, is performed subsequently to the method 900, such as after the user engages in multiple health experiences over time. Step 1002 generates an updated avatar for a user by converting a subsequent visual image of the user into a digital visual representation of the user. The avatar module 118, for example, captures a subsequent visual image of the user after the user engages in the health experience 220, e.g., multiple times over a period of time. The avatar module 118 converts the subsequent visual image into a digital visual representation of the user that reflects current physical attributes of the user, e.g., physical dimensions of the user. In at least one implementation, the health manager module 110 outputs the updated avatar, such as concurrently with the original avatar and the target avatar to provide a visual indication of differences between the avatars and/or a difference between physical attributes of the user upon which the original avatar is based and physical attributes of the user upon which the updated avatar is based.

Step 1004 determines health progress of the user by comparing the updated avatar to the original avatar and the target avatar to determine progress toward a change in physical attributes of the user. The health manager module 110, for example, compares visual dimensions of the avatars to determine whether the updated avatar indicates that the user has made progress toward a desired change in physical attributes indicated by the target avatar. For instance, in a scenario where the change in physical attributes indicates a reduction in body mass, the health manager module determines whether the updated avatar indicates a reduction in body mass, no change in body mass, or an increase in body mass. In at least one implementation, the health manager module 110 outputs an indication of whether progress is detected. For instance, the interaction module 114 outputs interaction content that indicates the progress toward the change in the one or more physical attributes of the user. Consider, for example, an implementation where the desired change in physical attributes includes a reduction in overall body mass. If the updated avatar reflects a reduction in body mass, the interaction module 114 outputs congratulatory content such as audio content indicating “good job, you've made progress toward your goal!”

Step 1006 generates updated health experience data based on the progress toward the change in the one or more physical attributes to include an updated exercise set. The health manager module 110, for instance, identifies an exercise set that is targeted to help the user progress from the physical attribute state indicated by the updated avatar to the physical attribute state indicated by the target avatar. For example, in an implementation where the updated avatar indicates little or no progress toward the target avatar, the health manager module 110 identifies an exercise set that is targeted to increase physical exertion of the user, e.g., to burn fat, build muscle mass, etc. The health manager module 110 adds the updated exercise set to a health experience, such as to replace or supplement an existing exercise and to generate an updated health experience.

Step 1008 outputs the updated health experience data including the updated exercise set. For example, the health manager module outputs the updated health experience data including outputting the updated exercise set as part of an overall health experience.

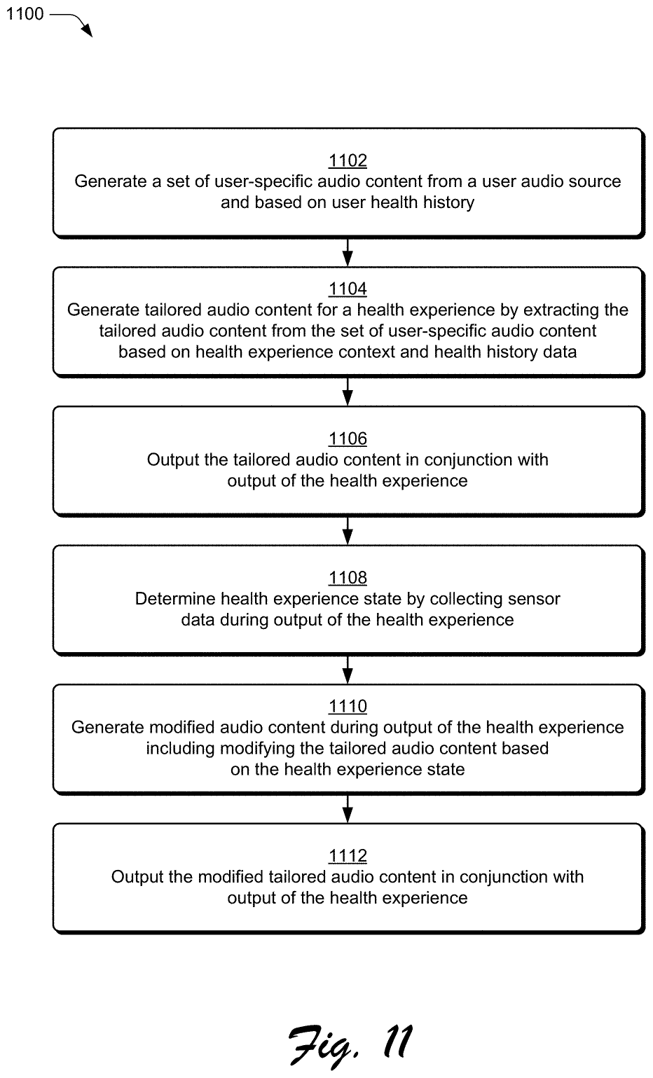

FIG. 11 depicts an example method 1100 for aggregating audio content for a health experience. Step 1102 generates a set of user-specific audio content from a user audio source and based on user health history. The audio module 116, for example, accesses the health history data 132 and/or the audio data 128 to determine audio preferences for the user, such as based on user selection of audio content in conjunction with historical health experiences and/or user actions indicating a preference for particular instances and/or types of audio content. The audio module 116 utilizes the audio preferences to aggregate the user-specific audio content from the user audio source.

Step 1104 generates tailored audio content for a health experience by extracting the tailored audio content from the set of user-specific audio content based on health experience context and health history data. The audio module 116, for example, determines a health experience context for a particular health experience, examples of which are discussed above. Further, the audio module 116 accesses the health history data 132 to determine user reaction to audio content in conjunction with historical health experiences, such as whether the user reacted favorably or disfavorably to particular instances and/or types of audio content being output as part of the historical health experiences. In at least one implementation the health history data 132 identifies specific instances of audio content (e.g., instances of music) that the user has selected and/or reacted favorably to. For instance, the user selects particular instances of audio content for playback in conjunction with a health experience. Alternatively or additionally, the user provides positive feedback during playback of a particular instance of audio content, and thus the audio content is tagged (e.g., as a favorite) in the health history data 132. In at least one implementation, the user also specifies a particular time during a health experience for playback of a specific instance of audio content. The audio module 116 aggregates audio content from the user-specific audio content that correlates to the health experience context and the health history data, such as based on audio tempo, genre, artist, specific instances of audio content, and so forth.

Step 1106 outputs the tailored audio content in conjunction with output of the health experience. The audio module 116, for instance, leverages the audio system 142 to output the tailored audio content. Step 1108 determines health experience state by collecting sensor data during output of the health experience. The audio module 116, for instance, monitors health experience state during output of the health experience, such as by receiving sensor data 146 during output of the health experience. Examples of health experience state are discussed above and include user reactions to a health experience such as facial expressions, body pose, exercise form and tempo, and so forth.

Step 1110 generates modified audio content during output of the health experience including modifying the tailored audio content based on the health experience state. The audio module 116, for example, determines that the tailored audio content is to be modified such as based on detecting user state from the health experience state. For example, the health experience state indicates that the user's exercise tempo is slowing and thus the audio module 116 modifies audio output to include more up-tempo audio than currently specified by the tailored audio content. As another example the health experience state indicates that the user is struggling with a current exercise (e.g., based on facial expression) and thus the audio module 116 modifies audio output to include more relaxing audio content, e.g., audio content with a slower tempo. In at least one implementation, the tailored audio content is modified to include a specific instance of audio content that is indicated as a favorite of the user, such as in the health history data 132. For instance, the instance of audio content is identified (e.g., via explicit and/or implicit user input) as being motivational to the user and thus is output to encourage the user during a difficult portion of the health experience. Step 1112 outputs the modified tailored audio content in conjunction with output of the health experience. The audio module 116, for instance, leverages the audio system 142 to output the modified tailored audio content. Accordingly, audio content is dynamically modifiable during a health experience to adapt to changes in user and/or environmental state.

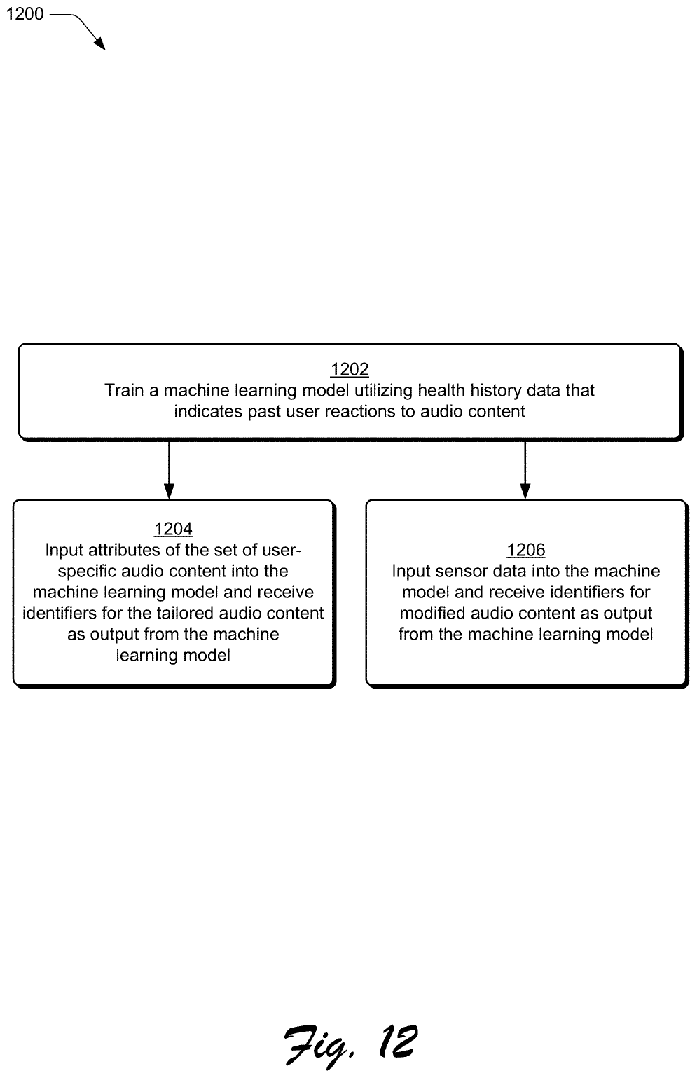

FIG. 12 depicts an example method 1200 for utilizing machine learning for audio content of a health experience. Step 1202 trains a machine learning model utilizing health history data that indicates past user reactions to audio content. The audio module 116, for example, includes and/or has access to a machine learning model that is trainable to predict various audio attributes. Accordingly, the audio module 116 trains the machine learning model utilizing health history data that indicates past user reactions to audio content as part of one or more historical health experiences. The past user reactions, for instance, represent positive and negative reactions to particular instances and/or types of audio content that were output in conjunction with the historical health experiences. Alternatively or additionally, the past user reactions indicate a change in exercise form detected from a user in conjunction with output of audio content during the one or more historical health experiences. For instance, a particular user reaction indicates an improvement in exercise form that coincided temporally with output of a particular instance and/or type of audio content.

Step 1204 inputs attributes of the set of user-specific audio content into the machine learning model and receives identifiers for the tailored audio content as output from the machine learning model. The audio module 116, for instance, utilizes the trained machine learning model to obtain tailored audio content for a health experience, such as for implementing aspects of step 1104 of the method 1100.

Alternatively or additionally to utilizing the trained machine learning model to generate tailored audio content, the trained machine learning model is usable to dynamically modify audio content during output of a health experience. For instance, the audio module 116 utilizes the trained machine learning model to implement aspects of steps 1108, 1110 of the method 1100. Step 1206 inputs sensor data into the machine model and receives identifiers for modified audio content as output from the machine learning model. The audio module 116, for example, receives sensor data 146 from the sensor system 138 and inputs the sensor data 146 into the trained machine learning model. The machine learning model outputs identifiers for audio content to be used to modify audio content being output during a health experience, e.g., the tailored audio content. Generally, this enables the audio module 116 to utilize machine learning techniques to dynamically adapt to changes in health experience state detected during output of a health experience, e.g., changes in user mood and/or exercise form that are detected in conjunction with a health experience.

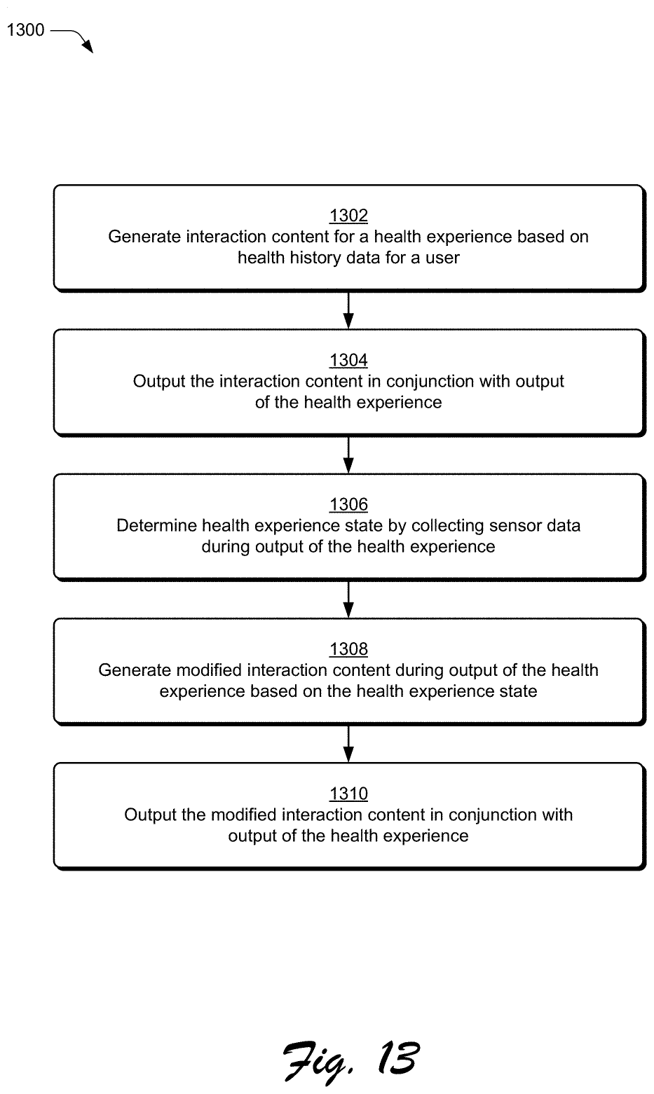

FIG. 13 depicts an example method 1300 for aggregating interaction content for a health experience. Step 1302 generates interaction content for a health experience based on health history data for a user. The interaction module 114, for instance, accesses the health history data 132 for a user and correlates the health history data 132 to interaction content for inclusion with a health experience. The health history data 132, for instance, includes past user reactions to particular instances and/or types of interaction content output as part of historical health experiences. Thus, the interaction module 114 identifies the interaction content based on the past user reactions, e.g., based on previous interaction content that occurred in conjunction with positive user reactions such as improved user mood and/or improved user participation in a health experience. Alternatively or additionally, the health history data 132 identifies particular exercises with which the user has historically struggled and/or time points during exercise sessions (e.g., health experiences 136) at which the user has struggled. Thus, the interaction module 114 generates interaction data to provide motivation and support in conjunction with the particular exercises and/or time points during a health experience 136.

Step 1304 outputs the interaction content in conjunction with output of the health experience. The interaction module 114, for instance, leverages the audio system 142 and/or the display device 140 to output the interaction content. Step 1306 determines health experience state by collecting sensor data during output of the health experience. The interaction module 114, for instance, monitors health experience state during output of the health experience, such as by receiving sensor data 146 during output of the health experience. Examples of health experience state are discussed above and include user reactions to a health experience such as facial expressions, body pose, exercise form and tempo, and so forth.

Step 1308 generates modified interaction content during output of the health experience based on the health experience state. The interaction module 114, for example, determines that the interaction content is to be modified such as based on detecting user state from the health experience state. For example, the health experience state indicates that the user's exercise tempo is slowing and thus the interaction module 114 generates interaction content to encourage the user to increase exercise tempo, e.g., “pick up the pace a bit, you're almost there!” As another example the health experience state indicates that the user is struggling with a current exercise (e.g., based on facial expression) and thus the interaction module 114 outputs interaction content to suggest that the user slow their exercise tempo, e.g., “slow down a bit, you're trying too hard!”

Step 1310 outputs the modified interaction content in conjunction with output of the health experience. The interaction module 114, for instance, leverages the audio system 142 and/or the display device 140 to output the modified interaction content. Accordingly, interaction content is dynamically modifiable during a health experience to adapt to changes in user and/or environmental state.

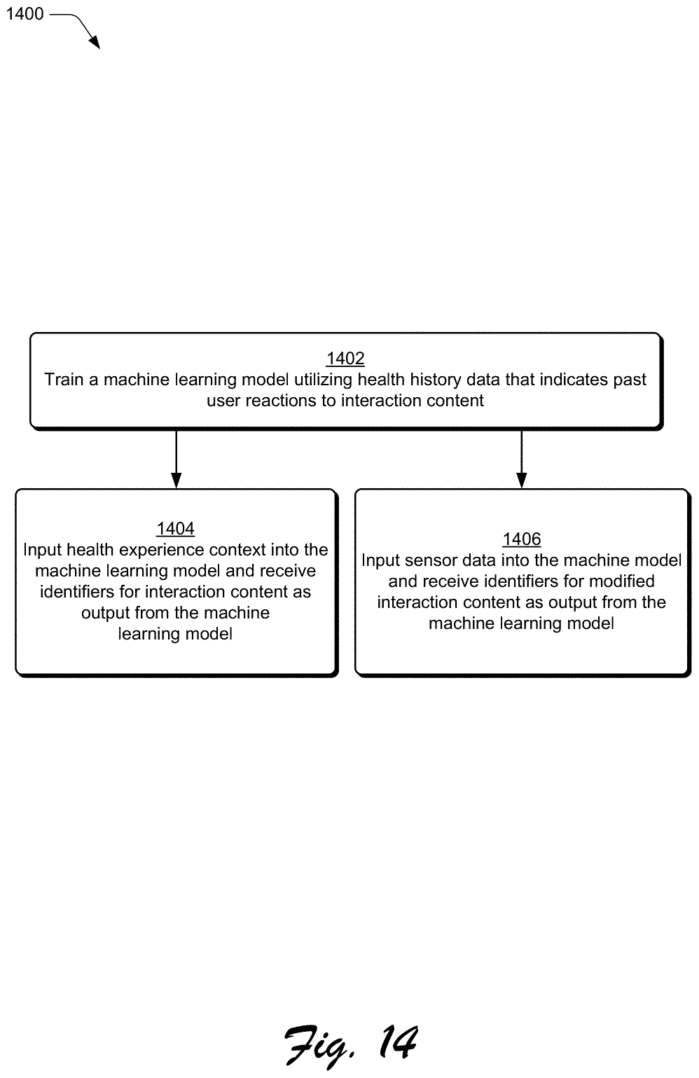

FIG. 14 depicts an example method 1400 for utilizing machine learning for interaction content of a health experience. Step 1402 trains a machine learning model utilizing health history data that indicates past user reactions to interaction content output in conjunction with particular exercises. The interaction module 114, for example, includes and/or has access to a machine learning model that is trainable to predict various interaction content attributes. Accordingly, the interaction module 114 trains the machine learning model utilizing health history data that indicates past user reactions to interaction content as part of one or more historical health experiences that include particular exercise types. The past user reactions, for instance, represent positive and negative reactions to particular instances and/or types of interaction content that were output in conjunction with particular exercise types for the historical health experiences. Alternatively or additionally, the past user reactions indicate a change in exercise form detected from a user in conjunction with output of interaction content during the one or more historical health experiences. For instance, a particular user reaction indicates an improvement in exercise form that coincided temporally with output of a particular instance and/or type of interaction content.

Step 1404 inputs health experience context data into the machine learning model and receives identifiers for interaction content as output from the machine learning model. For instance, health experience context data that identifies a particular exercise type is input into the trained machine learning model and the machine learning model predicts a subset of interaction content from the interaction data 126 that is likely to provide a user with a favorable health experience. The interaction module 114, for instance, utilizes the trained machine learning model to obtain interaction content for a health experience, such as for implementing aspects of step 1302 of the method 1300.

Alternatively or additionally the trained machine learning model is usable to dynamically modify interaction content during output of a health experience. For instance, the interaction module 114 utilizes the trained machine learning model to implement aspects of steps 1306, 1308 of the method 1300. Step 1406 inputs sensor data into the machine model and receives identifiers for modified interaction content as output from the machine learning model. The interaction module 114, for example, receives sensor data 146 from the sensor system 138 and inputs the sensor data 146 into the trained machine learning model. The machine learning model outputs identifiers for interaction content to be used to modify interaction content being output during a health experience, e.g., the modified interaction content. Generally, this enables the interaction module 114 to utilize machine learning techniques to dynamically adapt to changes in health experience state detected during output of a health experience, e.g., changes in user mood and/or exercise form that are detected in conjunction with a health experience.

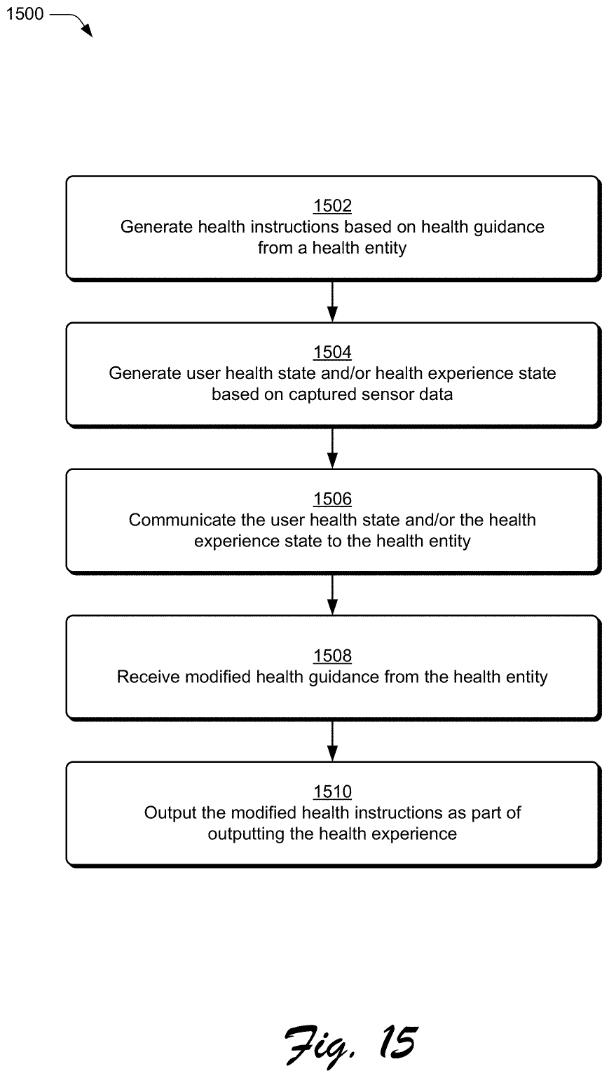

FIG. 15 depicts an example method 1500 for generating health instructions for a health experience. Step 1502 generates health instructions based on health guidance from a health entity. The health manager module 110, for instance, receives health guidance from the health entity 214 via the health interface module 120. In at least one implementation, the health guidance is received based on a previous interaction between the health manager system 102 and the health entity 214. For instance, the health manager module 110 aggregates user health status data for a user (examples of which are discussed above) and communicates the data to the health entity 214. The health entity 214 then generates health guidance based on the health status data, such as guidance for achieving a particular health goal.

The health manager module 110 converts the health guidance into health instructions for the health experience. For instance, the health manager module 110 identifies exercises and/or other movements that correlate to the health guidance, e.g., that are targeted to implement the health guidance. In at least one implementation, the health guidance identifies a suggested user movement for a health experience, and said converting the health guidance into the health instructions includes mapping the suggested user movement to an exercise that involves the suggested user movement. Accordingly, the health manager module 110 outputs the health experience including the health instructions.

Step 1504 generates user health state and/or health experience state based on captured sensor data. The health manager module 110, for instance, receives sensor data 146 from the sensor system 138 and correlates the sensor data 146 to the user health state and/or the health experience state. Generally, the user health state and/or the health experience state are generated at various points relative to a health experience, such as before output of a health experience, during output of a health experience, and/or after output of a health experience. Step 1506 communicates the user health state and/or the health experience state to the health entity. The health manager module 110, for instance, leverages the health interface module 120 to communicate the user health state and/or the health experience state to the health entity 214. In at least one implementation, the health entity 214 is implemented on a system that is remote from the health manager system 102, and thus the user health state and/or the health experience state are communicated over a network for receipt by the health entity 214.

Step 1508 receives modified health guidance from the health entity. The health interface module 120, for instance, receives modified health guidance from the health entity 214 and based on the user health state and/or the health experience state. Step 1510 generates modified health instructions based on the modified health guidance. The health manager module 110, for example, generates modified health instructions by converting the modified health guidance into the modified health instructions for output as part of the health experience.

Step 1510 outputs the modified health instructions as part of outputting the health experience. The health manager module, for example, outputs the health experience by outputting the modified health instructions. In at least one implementation, aspects of the method 1500 are performed in real time during output of a health experience such as to obtain modified health guidance from the health entity 214 for use in dynamically adapting the health experience based on detected changes in user health state and/or the health experience state.

Accordingly, techniques for dynamically adaptable health experience based on data triggers provide for dynamic and adaptable health experiences by leveraging a variety of different data and state conditions for generating and modifying health experiences.

The example methods described above are performable in various ways, such as for implementing different aspects of the systems and scenarios described herein. For instance, aspects of the methods are implemented by the health manager module 110 and various aspects of the methods are implemented via the different GUIs described above. Generally, any services, components, modules, methods, and/or operations described herein are able to be implemented using software, firmware, hardware (e.g., fixed logic circuitry), manual processing, or any combination thereof. Some operations of the described methods, for example, are described in the general context of executable instructions stored on computer-readable storage memory that is local and/or remote to a computer processing system, and implementations include software applications, programs, functions, and the like. Alternatively or in addition, any of the functionality described herein is performable, at least in part, by one or more hardware logic components, such as, and without limitation, Field-programmable Gate Arrays (FPGAs), Application-specific Integrated Circuits (ASICs), Application-specific Standard Products (ASSPs), System-on-a-chip systems (SoCs), Complex Programmable Logic Devices (CPLDs), and the like. The order in which the methods are described is not intended to be construed as a limitation, and any number or combination of the described method operations are able to be performed in any order to perform a method, or an alternate method.

Having described example procedures in accordance with one or more implementations, consider now an example system and device that are able to be utilized to implement the various techniques described herein.

Example System and Device

FIG. 16 illustrates an example system 1600 that includes an example computing device 1602 that is representative of one or more computing systems and/or devices that are usable to implement the various techniques described herein. This is illustrated through inclusion of the health manager module 110. The computing device 1602 includes, for example, a server of a service provider, a device associated with a client (e.g., a client device), an on-chip system, and/or any other suitable computing device or computing system.

The example computing device 1602 as illustrated includes a processing system 1604, one or more computer-readable media 1606, and one or more I/O interfaces 1608 that are communicatively coupled, one to another. Although not shown, the computing device 1602 further includes a system bus or other data and command transfer system that couples the various components, one to another. For example, a system bus includes any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1604 is representative of functionality to perform one or more operations using hardware. Accordingly, the processing system 1604 is illustrated as including hardware elements 1610 that are be configured as processors, functional blocks, and so forth. This includes example implementations in hardware as an application specific integrated circuit or other logic device formed using one or more semiconductors. The hardware elements 1610 are not limited by the materials from which they are formed or the processing mechanisms employed therein. For example, processors are comprised of semiconductor(s) and/or transistors (e.g., electronic integrated circuits (ICs)). In such a context, processor-executable instructions are, for example, electronically-executable instructions.

The computer-readable media 1606 is illustrated as including memory/storage 1612. The memory/storage 1612 represents memory/storage capacity associated with one or more computer-readable media. In one example, the memory/storage 1612 includes volatile media (such as random access memory (RAM)) and/or nonvolatile media (such as read only memory (ROM), Flash memory, optical disks, magnetic disks, and so forth). In another example, the memory/storage 1612 includes fixed media (e.g., RANI, ROM, a fixed hard drive, and so on) as well as removable media (e.g., Flash memory, a removable hard drive, an optical disc, and so forth). The computer-readable media 1606 is configurable in a variety of other ways as further described below.

Input/Output interface(s) 1608 are representative of functionality to allow a user to enter commands and information to computing device 1602, and also allow information to be presented to the user and/or other components or devices using various Input/Output devices. Examples of input devices include a keyboard, a cursor control device (e.g., a mouse), a microphone, a scanner, touch functionality (e.g., capacitive or other sensors that are configured to detect physical touch), a camera (e.g., which employs visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1602 is configurable in a variety of ways as further described below to support user interaction.

Various techniques are described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques are implementable on a variety of commercial computing platforms having a variety of processors.

Implementations of the described modules and techniques are storable on or transmitted across some form of computer-readable media. For example, the computer-readable media includes a variety of media that that is accessible to the computing device 1602. By way of example, and not limitation, computer-readable media includes “computer-readable storage media” and “computer-readable signal media.”

“Computer-readable storage media” refers to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which are accessible to a computer.

“Computer-readable signal media” refers to a signal-bearing medium that is configured to transmit instructions to the hardware of the computing device 1602, such as via a network. Signal media typically embodies computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1610 and computer-readable media 1606 are representative of modules, programmable device logic and/or fixed device logic implemented in a hardware form that is employable in some embodiments to implement at least some aspects of the techniques described herein, such as to perform one or more instructions. Hardware includes components of an integrated circuit or on-chip system, an application-specific integrated circuit (ASIC), a field-programmable gate array (FPGA), a complex programmable logic device (CPLD), and other implementations in silicon or other hardware. In this context, hardware operates as a processing device that performs program tasks defined by instructions and/or logic embodied by the hardware as well as a hardware utilized to store instructions for execution, e.g., the computer-readable storage media described previously.

Combinations of the foregoing are also employable to implement various techniques described herein. Accordingly, software, hardware, or executable modules are implementable as one or more instructions and/or logic embodied on some form of computer-readable storage media and/or by one or more hardware elements 1610. For example, the computing device 1602 is configured to implement particular instructions and/or functions corresponding to the software and/or hardware modules. Accordingly, implementation of a module that is executable by the computing device 1602 as software is achieved at least partially in hardware, e.g., through use of computer-readable storage media and/or hardware elements 1610 of the processing system 1604. The instructions and/or functions are executable/operable by one or more articles of manufacture (for example, one or more computing devices 1602 and/or processing systems 1604) to implement techniques, modules, and examples described herein.

The techniques described herein are supportable by various configurations of the computing device 1602 and are not limited to the specific examples of the techniques described herein. This functionality is also implementable entirely or partially through use of a distributed system, such as over a “cloud” 1614 as described below.

The cloud 1614 includes and/or is representative of a platform 1616 for resources 1618. The platform 1616 abstracts underlying functionality of hardware (e.g., servers) and software resources of the cloud 1614. For example, the resources 1618 include applications and/or data that are utilized while computer processing is executed on servers that are remote from the computing device 1602. In some examples, the resources 1618 also include services provided over the Internet and/or through a subscriber network, such as a cellular or Wi-Fi network.

The platform 1616 abstracts the resources 1618 and functions to connect the computing device 1602 with other computing devices. In some examples, the platform 1616 also serves to abstract scaling of resources to provide a corresponding level of scale to encountered demand for the resources that are implemented via the platform. Accordingly, in an interconnected device embodiment, implementation of functionality described herein is distributable throughout the system 1600. For example, the functionality is implementable in part on the computing device 1602 as well as via the platform 1616 that abstracts the functionality of the cloud 1614.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. A method implemented by at least one computing device for generating health experience data for a user profile, the method comprising:
generating, by an avatar module, an original avatar for a user by capturing a visual image of the user and converting the visual image into a digital visual representation of the user that reflects physical attributes of the user;
generating, by the avatar module, a target avatar by receiving user input to manipulate one or more visual features of the original avatar and adjusting a visual appearance of the original avatar based on the one or more manipulated visual features;
generating, by a health manager module, health experience data by comparing the target avatar to the original avatar, determining a visual difference between the target avatar and the original avatar, correlating the visual difference to a corresponding change in one or more physical attributes of the user, and generating the health experience data to include an exercise set targeted to achieve the corresponding change in the one or more physical attributes; and
outputting, by the health manager module, the health experience data including outputting the exercise set.

2. A method as described in claim 1, wherein the original avatar includes a representation of a physical dimension of the user, and the one or more manipulated visual features comprise a manipulation of the representation of the physical dimension of the user to generate the target avatar.

3. A method as described in claim 2, wherein the visual difference between the target avatar and the original avatar comprises a difference in the representation of the physical dimension of the user, the change in the one or more physical attributes of the user comprises a change in the physical dimension of the user, and the exercise set is targeted to achieve the change in the physical dimension of the user.

4. A method as described in claim 1, wherein said generating the health experience data comprises comparing, by the health manager module, the change in one or more physical attributes of the user to exercise data that describes multiple different exercises and mapping the change in one or more physical attributes to the exercise set from the exercise data.

5. A method as described in claim 1, further comprising:
generating, by the avatar module and subsequent to said outputting the health experience data, an updated avatar for the user by capturing a subsequent visual image of the user and converting the subsequent visual image into a digital visual representation of the user that reflects current physical attributes of the user; and
outputting, by the health manager module, the original avatar, the updated avatar, and the target avatar.

6. A method as described in claim 5, further comprising:
determining, by the health manager module, health progress of the user by comparing the updated avatar to the original avatar and the target avatar to determine progress toward the change in the one of more physical attributes of the user; and
outputting, by an interaction module, interaction content that indicates the progress toward the change in the one or more physical attributes of the user.

7. A method as described in claim 6, further comprising:
generating, by the health manager module and based on the progress toward the change in the one or more physical attributes of the user, updated health experience data to include an updated exercise set; and
outputting, by the health manager module, the updated health experience data including outputting the updated exercise set.

8. In a digital environment for health management, a system comprising:
an audio module implemented at least partially in hardware of at least one computing device to generate tailored audio content for a health experience including to determine a health experience context for a health experience and extract the tailored audio content from a set of user-specific audio content based on the health experience context;
an interaction module implemented at least partially in the hardware of the at least one computing device to generate interaction content for output as part of the health experience including to determine health history data for a user and correlate the health history data to the interaction content;
a health manager module implemented at least partially in the hardware of the at least one computing device to: output the health experience including at least some of the tailored audio content and at least some of the interaction content; determine health experience state by collecting sensor data during output of the health experience; and to perform one or more of to:
implement the audio module to generate modified audio content during output of the health experience including to modify the tailored audio content based on the health experience state; or
implement the interaction module to generate modified interaction content during output of the health experience including to modify the interaction content based on the health experience state.

9. A system as described in claim 8, wherein the audio module is further implemented to generate the set of user-specific audio content including to determine user audio preferences based on health history data for the user, and to extract the user-specific audio content from a user audio source based on the user audio preferences.

10. A system as described in claim 8, wherein the health experience context comprises one or more of an exercise or an exercise parameter for the health experience, and wherein the audio module is implemented to extract the tailored audio content based at least in part on the one or more of the exercise or the exercise parameter.

11. A system as described in claim 8, wherein the audio module is implemented to:
train a machine learning model utilizing at least a portion of the health history data that indicates past user reactions to audio content as part of one or more historical health experiences; and
input attributes of the set of user-specific audio content into the machine learning model and receive identifiers for the tailored audio content as output from the machine learning model.

12. A system as described in claim 11, wherein the past user reactions comprise a change in exercise form detected from a user in conjunction with output of audio content during the one or more historical health experiences.

13. A system as described in claim 8, wherein the health history data comprises past user reactions to interaction content output as part of historical health experiences, and wherein the interaction module is implemented to generate the interaction content based on the past user reactions.

14. A system as described in claim 8, wherein the health experience context identifies a particular exercise type for an exercise included in the health experience, and wherein the interaction module is further implemented to:
train a machine learning model using at least a portion of the health history data that indicates past user reaction to interaction content that was output in conjunction with the particular exercise type; and
input the exercise type into the machine learning model and receive an indication of at least some of the interaction content for the health experience as output from the machine learning model.

15. A system as described in claim 8, wherein the health experience state comprises one or more of a facial expression or a user movement attribute detected via the sensor data in conjunction with output of the health experience.

16. A system as described in claim 8, wherein the health manager module is implemented to:
train a machine learning model utilizing one or more of:
at least a portion of the health history data that indicates past user reactions to audio content as part of one or more historical health experiences; or
at least a portion of the health history data that indicates past user reaction to interaction content that was output in conjunction with a particular exercise type; and
to perform one or more of to:
modify the tailored audio content based on the health experience state including to input the sensor data into the trained machine learning model and receive the modified audio content as output from the machine learning model; or
modify the interaction content including to input the sensor data into the trained machine learning model and receive the modified interaction content as output from the machine learning model.

17. A method implemented by at least one computing device for generating health instructions for a health experience, the method comprising:
generating, by a health manager module, health instructions for a health experience by receiving health guidance from a health entity and converting the health guidance into the health instructions for output as part of the health experience;
generating, by the health manager module, one or more of user health state or health experience state by capturing sensor data, correlating the sensor data to the one or more of the user health state or the health experience state, and communicating the one or more of the user health state or the health experience state to the health entity;
receiving, by health interface module, modified health guidance from the health entity and based on the one or more of the user health state or the health experience state;
generating, by the health manager module, modified health instructions by converting the modified health guidance into the modified health instructions for output as part of the health experience; and
outputting, by the health manager module, the health experience by outputting at least some of the modified health instructions.

18. A method as described in claim 17, wherein the health guidance is received from a first system that is implemented remotely from a second system on which the health manager module is implemented.

19. A method as described in claim 17, wherein the sensor data is captured while the health experience is being output and the modified health instructions are implemented to modify output of the health experience while the health experience is being output.

20. A method as described in claim 17, wherein the health guidance identifies a suggested user movement for the health experience, and said converting the health guidance into the health instructions comprises mapping the suggested user movement to an exercise that involves the suggested user movement.
